(19) United States
(12) Patent Application Publication
(10) Pub. No.: US 2024/0366327 A1
(43) Pub. Date: Nov. 7, 2024

(54) ROBOTIC SURGICAL SYSTEM AND RELATED METHODS

(71) Applicant: NuVasive, Inc., San Diego, CA (US)

(72) Inventors: David Berman, Solana Beach, CA (US); Amar Bhatt, San Diego, CA (US); Antonio Ubach, Tucson, AZ (US); Cara Lee Coad, Longmont, CO (US); Chasen Peters, La Mesa, CA (US); Jeremiah Beers, Evergreen, CO (US); Navid Mahpeykar, San Diego, CA (US); Patrick Digmann, Louisville, CO (US); Todd Baxendale, Broomfield, CO (US)

(21) Appl. No.: 18/625,900

(22) Filed: Apr. 3, 2024

Related U.S. Application Data
(60) Provisional application No. 63/494,342, filed on Apr. 5, 2023.

(51) Int. Cl. A61B 34/00; A61B 34/20; A61B 34/30

(52) U.S. Cl. CPC A61B 34/70; A61B 34/25; A61B 2034/2046; A61B 2034/302

(57) ABSTRACT

Examples described herein are relevant to robotic surgical systems, such as those used in spine surgery. Examples described herein include: a distal section of a robot arm, pods having fiducials, face switching angles, fiducial hollows, drape anchoring and sensing, selective face switching with active fiducials, pedal-less workflow, user interface control, hand guiding, robot egress, robot tool center point adjustment, collision reaction, dynamic screw placement ordering, flexible robot cart placement, depth gauges, implant checking, implant-to-instrument-checking, robot bed-side docking, workflow based cart immobilization, patient gross movement monitoring, selective brake control, auto vertical adjustment, gesture-based planning, automatic sleeve retention and retraction, among others.

### CROSS REFERENCE TO RELATED APPLICATIONS

This application is a non-provisional application which claims priority to provisional application Ser. No. 63/494,342, filed Apr. 5, 2023, which is incorporated in its entirety herein for all purposes.

This application is related to U.S. patent application Ser. No. 17/696,203, which was filed Mar. 16, 2022, and which is incorporated herein by reference in its entirety for any and all purposes. That application discloses systems and methods for using a robotic surgical system comprising a graphical user interface and a robotic arm.

This application is related to U.S. patent application Ser. No. 17/461,342, which was filed Aug. 30, 2021, and which is incorporated herein by reference in its entirety for any and all purposes. That application describes a robotic cart stabilization device. An example cart is a surgical cart having a robotic arm thereon. A stabilizer system may be part of or used with the cart to stabilize the cart at a location. The stabilizer system may include a stabilizer and an actuator. The stabilizer may have a foot and a biaser configured to bias the foot to a retracted position and contribute to an amount of force applied to a floor supporting the cart when the foot is in a deployed position. The actuator acts on the stabilizer to overcome a bias force biasing the stabilizer to the retracted position and cause feet of the stabilizer to contact the floor. Once the feet of the stabilizer contact the floor, a spring of the biaser causes the foot to apply a predetermined force amount to the floor.

This application is related to U.S. patent application Ser. No. 17/688,574, which was filed Mar. 7, 2022, and which is incorporated herein by reference in its entirety for any and all purposes. That application describes a surgical cart that includes a local client access point, a monitor, a cart computer, a first external interface coupled to the local client access point, and a second external interface coupled to the primary monitor. The surgical cart can be for use in an operating room to provide multiple different surgical applications (e.g., navigation, neuromonitoring, surgical planning, imaging, rod bending, and robot control) from the cart. Generally, the cart is a portable unit configured to be readily movable within or between operating rooms usable during an operation to interact with one or more of the multiple surgical applications (e.g., by providing the cart with lockable wheels). In an example, the cart includes a computer, output devices (e.g., a monitor and speaker), input devices (e.g., a touchscreen of the monitor and a keyboard), and connections for other devices (e.g., a network connection, an imaging device connection, and a navigation camera connection). The surgical cart can host a web server to which one or more client devices can connect using a web browser. The web server can provide access to one or more surgical applications hosted by the surgical cart during a surgery.

This application is related to U.S. patent application Ser. No. 17/537,651, which was filed Nov. 30, 2021, and which is incorporated herein by reference in its entirety for any and all purposes. That application describes robotically controlled laser or ultrasonic instruments used to remove tissue during a surgery, such as to form one or more pilot holes in a vertebra or a window in bone. Where a laser is used, interrogative laser pulses can be used to obtain information, such as detecting depth or tissue type.

This application relates to U.S. patent application Ser. No. 17/550,318, which was filed Dec. 14, 2021, and which is incorporated herein by reference in its entirety for any and all purposes. That application describes motion controlled robotic surgery. An image of a surgical site of a patient is obtained. The method includes capturing motion data of an instrument based on a user-defined path to the surgical site. The method includes determining a motion path based on the captured motion data. The motion path corresponds to an actuation of one or more components of a robotic device. The method includes displaying a portion of the motion path onto the image. The method includes determining one or more instructions for actuating the one or more components along the determined motion path. The method includes providing the one or more instructions to the robotic device.

This application relates to U.S. patent application Ser. No. 17/400,888, which was filed Aug. 12, 2021, and which is incorporated herein by reference in its entirety for any and all purposes. This application describes medical devices and connector assemblies for connecting medical devices. An example connector assembly for connecting a robotic arm with a medical end effector may include a plate configured for coupling to a robotic arm. The plate may have a connection region that includes a flange defining a circumferential groove. The connector assembly may also include an attachment assembly having an attachment region configured to be detachably secured to the connection region with a securing distance of less than 12 millimeters. The attachment region may include one or more engagement members that are configured to shift between an unsecured position and a secured position where the engagement members are secured to the connection region. An actuator may be coupled to the attachment assembly for shifting the one or more engagement members between the unsecured position and the secured position.

This application relates to U.S. Pat. No. 11,135,015, filed Jul. 17, 2018, as application Ser. No. 16/037,175. That patent is incorporated herein by reference in its entirety for any and all purposes. That patent describes a surgical implant-planning computer for intra-operative and pre-operative imaging workflows. A network interface is connectable to an imager and a robot surgical platform having a robot base coupled to a robot arm that is movable by motors. An image of a bone is received from the imager and displayed. A user's selection is received of a surgical screw from among a set of defined surgical screws. A graphical screw representing the selected surgical screw is displayed as an overlay on the image of the bone. Angular orientation and location of the displayed graphical screw relative to the bone in the image is controlled responsive to receipt of user inputs. An indication of the selected surgical screw and an angular orientation and a location of the displayed graphical screw are stored in a surgical plan data structure.

### BACKGROUND

A wide variety of surgical devices and systems have been developed for medical use, for example, surgical use. Some of these devices and systems include carts and robots for use in surgical procedures, among other devices and systems. These devices and systems are manufactured by any one of a variety of different manufacturing methods and may be used according to any one of a variety of methods. Of the known surgical devices, systems, and methods, each has certain advantages and disadvantages. There is an ongoing desire for alternative surgical devices and systems, as well as alternative methods for manufacturing and using the surgical devices and systems.

### SUMMARY

Robotic surgery or robot-assisted surgery can allow surgeon to perform complex surgical procedures. Such techniques are relevant to improvements to precision, flexibility, and control. In some circumstances, using the robotic arm, surgeons may be able to perform delicate and complex procedures that would be difficult or impossible with traditional methods. However, surgeries involving a robotic arm may present various challenges. Hardware and/or software in the art can benefit from examples described herein, which may be used to provide improvements for and/or reduce drawbacks of existing systems. While many examples are described herein in the spine context, the examples described herein may be modified to be used in other contexts, such as in arthroplasty procedures, cranial procedures, other procedures, or combinations thereof.

Examples described herein can be implemented in a manner that improves existing robotic surgical systems. Examples described herein include: a distal section of a robot arm, pods having fiducials, face switching angles, fiducial hollows, drape anchoring and sensing, selective face switching with active fiducials, pedal-less workflow, user interface control, hand guiding, robot egress, robot tool center point adjustment, collision reaction, dynamic screw placement ordering, flexible robot cart placement, depth gauges, implant checking, implant-to-instrument-checking, robot bed-side docking, workflow based cart immobilization, patient gross movement monitoring, selective brake control, auto vertical adjustment, gesture-based planning, automatic sleeve retention and retraction, among others.

### BRIEF DESCRIPTION OF THE DRAWINGS

Many advantages of the present invention will be apparent to those skilled in the art with a reading of this specification in conjunction with the attached drawings, wherein like references are applied to like elements and wherein:

FIG. 1 illustrates an example system for performing a surgical procedure.

FIG. 2 illustrates an example robotic device that may be used during a surgical procedure.

FIG. 3 illustrates an example block diagram of a computing device.

FIG. 4 illustrates an example computer medium.

FIG. 5 illustrates a distal section of a robot arm having a tracking section, a user interface section, and a tool connector section.

FIGS. 6A-C illustrate pods of a robot arm having fiducials disposed within hollows.

FIG. 6D illustrates a front view of an end effector having an end effector circuit board and circuit boards of three pods installed in the end effector with all other components removed for ease of viewing the relative location of the circuit boards.

FIG. 7A illustrates a surface having a fiducial disposed relative to a hollow in the surface.

FIG. 7B annotates the surface, fiducial and hollow of FIG. 7A.

FIG. 7C illustrates an example of a shallow hollow in which the fiducial is only partially sunk beneath the surface.

FIG. 7D illustrates a surface having a plurality of hollows with each hollow having a fiducial disposed therein.

FIG. 7E illustrates a camera being used to track the locations of the fiducials of the hollows of the surface of FIG. 7D and the second face is oriented towards the camera.

FIG. 7F illustrates a camera being used to track the locations of the fiducials of the hollows of the surface of FIG. 7D and the first face is oriented towards the camera.

FIG. 7G illustrates a camera being used to track the locations of the fiducials of the hollows of the surface of FIG. 7D and the fourth face is oriented towards the camera.

FIG. 7H illustrates a partial transparency view of the housings of three pods with lines showing cones of visibility for each fiducial limited by their respective hollow.

FIG. 7I illustrates a simplified view of the cones of visibility for each of the pods of FIG. 7H.

FIG. 7J illustrates a method for creating a surface having hollows.

FIG. 8A illustrates a body covered by a drape.

FIG. 8B illustrates the anchor disposed within a capture area of the body.

FIG. 8C illustrates a partial cross-section view of the capture area taken along line C-C of FIG. 8B.

FIG. 9A illustrates a surgical tool having a controller, a first face, a second face, and a third face.

FIG. 9B illustrates a chart of example signals over the course of three frames (n, n+1, and n+2).

FIG. 9C illustrates the surgical tool during and shortly after the first frame.

FIG. 9D illustrates the surgical tool during and shortly after the second frame.

FIG. 9E illustrates the surgical tool during and shortly after the third frame.

FIG. 10 illustrates an example method.

FIG. 11 depicts a connector assembly, which generally can be used for connecting or otherwise securing a robotic arm and a medical end effector.

FIG. 12 illustrates a surgical navigation system configured to facilitate parking a robotic cart relative to a patient.

FIG. 13. illustrates example placements of a robot relative to a patient in the prone and lateral positions.

FIG. 14A illustrates a display providing a first user interface showing a representation of a vertebra having a representation of an instrument and an implant disposed relative thereto.

FIG. 14B illustrates the display providing a second user interface showing a representation of the vertebra.

FIG. 15A-15C illustrate a surgical system including a surgical navigation system having an implant confirmation algorithm for confirming the appropriateness of an implant coupled to an instrument and placed in a reference.

FIG. 15D illustrates an example algorithm for implant confirmation. The algorithm can start with operation.

FIG. 16A illustrates a robot arm having a robot end effector coupled thereto and disposed proximate a vertebra.

FIG. 16B illustrates a cross section view of FIG. 16A showing a robot end effector, sleeve, button, and instrument.

FIG. 16C illustrates an enlarged view of a button region of FIG. 16B.

### DETAILED DESCRIPTION

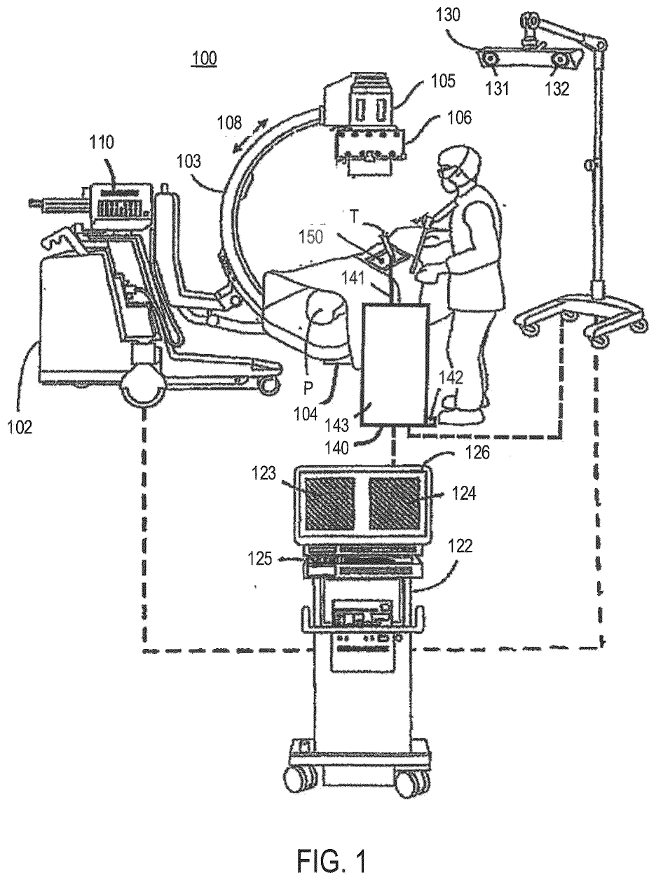

Disclosed examples related to improvements to surgical systems, such as those involving the use of robotic system to facilitate surgery. An example robot system that can benefit from technologies herein is described in relation to FIGS. 1-4.

Referring now to the figures, FIG. 1 is a diagram of an example system 100 for performing a surgical procedure. The example system 100 includes a base unit 102 supporting a C-Arm imaging device 103. The C-Arm 103 includes a radiation source 104 that is positioned beneath the patient P and that directs a radiation beam upward to the receiver 105. The receiver 105 of the C-Arm 103 transmits image data to a processing device 122. The processing device 122 may communicate with a tracking device 130 to obtain position and orientation information of various instruments T and H used during the surgical procedure, including the position and orientation of one or more portions of the robotic device 140. In addition to or instead of the term “tracking”, other terms can be used to refer to such determination of the position and orientation of tracked objects, such as “navigation” or “surgical navigation”. The tracking device 130 may communicate with a robotic device 140 to provide location information of various tracking elements, such as marker 150. The robotic device 140 and the processing device 122 may communicate via one or more communication channels.

The C-Arm 103 can be implemented in any of a variety of ways. An example implementation includes those described in U.S. Pat. Nos. 10,573,023; 10,842,453; 10,849,580; 11,058,378; 11,100,668; 11,253,216; 11,523,785; and 11,523,833.

The base unit 102 includes a control panel 110 through which a user can control the location of the C-Arm 103, as well as the radiation exposure. The control panel 110 thus permits the radiology technician to “shoot a picture” of the surgical site at a surgeon's direction, control the radiation dose, and initiate a radiation pulse image.

The C-Arm 103 may be rotated about the patient P in the direction of the arrow 108 for different viewing angles of the surgical site. In some instances, implants or instruments T and H may be situated at the surgical site, necessitating a change in viewing angle for an unobstructed view of the site. Thus, the position of the receiver relative to the patient P. and more particularly relative to the surgical site of interest, may change during a procedure. Consequently, the receiver 105 may include a tracking target 106 mounted thereto that allows tracking of the position of the C-Arm 103 using the tracking device 130. By way of example only, the tracking target 106 may include a plurality of infrared (IR) reflectors or active IR emitters (which can be referred to as fiducials) spaced around the target, while the tracking device 130 is configured to triangulate the position of the receiver 105 from the IR signals reflected or emitted by the tracking target 106.

The processing device 122 can include electronic memory associated therewith and a processor for executing digital and software instructions. The processing device 122 may also incorporate a frame grabber that uses frame grabber technology to create a digital image for projection as displays 123 and 124 on a display device 126. The displays 123 and 124 are positioned for interactive viewing by the surgeon during the procedure. The two displays 123 and 124 may be used to show images from two views, such as lateral and A/P, or may show a baseline scan and a current scan of the surgical site, or a current scan and a “merged” scan based on a prior baseline scan and a low radiation current scan, as described herein. An input device 125, such as a keyboard or a touch screen, can allow the surgeon to select and manipulate the on-screen images. It is understood that the input device may incorporate an array of keys or touch screen icons corresponding to the various tasks and features implemented by the processing device 122. The processing device 122 includes a processor that converts the image data obtained from the receiver 105 into a digital format. In some cases, the C-Arm 103 may be operating in the cinematic exposure mode and generating many images each second. In these cases, multiple images can be averaged together over a short time period into a single image to reduce motion artifacts and noise.

The tracking device 130 includes sensors 131 and 132 for determining location data associated with a variety of elements (e.g., an infrared reflector or emitter) used in a surgical procedure. In one example, the sensors 131 and 132 may be a charge-coupled device (CCD) image sensor. In another example, the sensors 131 and 132 may be a complementary metal-oxide-semiconductor (CMOS) image sensor. It is also envisioned that a different number of other image sensors may be used to achieve the functionality described.

In one aspect of the present invention, the robotic device 140 may assist with holding an instrument T relative to the patient P during a surgical procedure. In one scenario, the robotic device 140 may be configured to maintain the instrument T in a relative position to the patient P as the patient P moves (e.g., due to breathing) or is moved (e.g., due to manipulation of the patient's body) during the surgical procedure. The robotic device 140 may assist by, for example, maintaining a guide tube in a rigid position to maintain a desired trajectory relative to anatomy of the patient P.

The robotic device 140 may include a robot arm 141, a pedal 142, and a mobile housing 143. The robotic device 140 may also be in communication with a display such as display 126. The robotic device 140 may also include a fixation device to fix the robotic device 140 to an operating table.

In one example, in a first tracking mode, the tracking device 130 is configured to capture motion data of a handheld instrument based on a user-defined path at a surgical target site of the patient. In this example, the processing device 122 determines a motion path corresponding to the captured motion data. Further, the processing device 122 determines one or more instructions for actuating the one or more components of the robotic device 140 along the determined motion path and provides the one or more instructions to the robotic device 140. In a second tracking mode, the tracking device 130 is configured to capture the pose (e.g., the orientation and position) of a handheld instrument based on a user-defined placement of the instrument at a surgical site of the patient. In this example, the processing device 122 determines a motion path corresponding to the captured pose of the instrument. As described with the first tracking mode, the processing device 122 determines one or more instructions for actuating the one more components of the robotic device 140 along the determined motion and provides the one or more instructions to the robotic device 140.

In one example, a user may control actuation of the robot arm 141 using a robot pedal 142. In one embodiment, the user may depress the robot pedal 142 to activate one or more modes of the robotic device 140. In one scenario, the user may depress the robot pedal 142 to allow the user to manually position the robot arm 141 according to a desired position. In another scenario, the user may depress the robot pedal 142 to activate a mode that enables the robot arm 141 to place an instrument T in a position according to a determined motion path as described above. In another scenario, the user may depress the robot pedal 142 to stop the robot arm 141 from proceeding with any further movements.

The robot arm 141 may be configured to receive one or more end effectors depending on the surgical procedure and the number of associated joints. In one example, the robot arm 141 may be a six joint arm. In this example, each joint includes an encoder that measures its angular value. The movement data provided by the one or more encoders, combined with the known geometry of the six joints, may allow for the determination of the position of the robot arm 141 and the position of the instrument T coupled to the robot arm 141. It also envisioned that a different number of joints may be used to achieve the functionality described herein.

The mobile housing 143 ensures easy handling of the robotic device 140 using wheels or handles or both. In one embodiment, the mobile housing 143 may include immobilization pads or an equivalent device. The mobile housing 143 may also include a control unit which provides one or more commands to the robot arm 141 and allows a surgeon to manually input data through the use of an interface, such as a touch screen, a mouse, a joystick, a keyboard or similar device.

In one example, the processing device 122 is configured to capture a pose of an instrument H (e.g., a portable instrument) via the tracking device 130. The captured pose of the instrument includes a combination of position information and orientation information. In this example, the pose of the instrument H is based on a user defined placement at a surgical site of the patient P. The user-defined placement is based on movement of the instrument H by a surgeon. In one scenario, the portable instrument comprises one or more infrared reflectors or emitters. Continuing with this example, the processing device 122 is configured to determine a motion path corresponding to the captured pose of the instrument H. The motion path is associated with the actuation of one or more components (e.g., one or more links and joints) of the robotic device 140. The processing device 122 is configured to determine one or more instructions for actuating the one or more components of the robotic device 140 along the determined motion path. Further, the processing device 122 is configured to provide the one or more instructions to the robotic device 140.

In another example, the processing device 122 is configured to compare a position of the one or more infrared reflectors of the portable instrument to a position of one or more infrared reflectors coupled to the patient. Based on the comparison, the processing 122 is configured to determine whether a distance between the one or more infrared reflectors of the portable instrument and the one or more infrared reflectors coupled to the patient is within a safety threshold. Based on the determination of the distance between the portable instrument and the patient, the processing device 122 is configured to determine one or more adjustments to the actuation of the one or more components of the robotic device.

In another example, the processing device 122 is configured to compare a position of the one or more infrared reflectors of the portable instrument to a position of one or more infrared reflectors coupled to the patient. Based on the comparison, the processing 122 is configured to determine whether a distance between the one or more infrared reflectors of the portable instrument and the one or more infrared reflectors coupled to the patient is within a safety threshold. Based on the determination of the distance between the portable instrument and the patient, the processing device 122 is configured to determine one or more adjustments to the actuation of the one or more components of the robotic device.

In another example, the processing device 122 is configured to provide an image of the surgical site for display on display device 126. In this example, the processing device 122 is configured to overlay at least a portion of the determined motion path onto the image of the surgical site. Overlaying at least a portion of the determined path would allow a surgeon to review the path and ensure that it aligns with a surgical operative plan. In one example, the processing device 122 may be further configured to receive an input from the surgeon confirming that the overlaid portion of the determined motion path aligns with the surgical operative plan. In one example, the input is received through input device 125.

In another example, the processing device 122 is configured to determine an angle between a preoperative trajectory and the trajectory corresponding to the portion of the motion path. Based on the determined angle, the processing device 122 is configured to determine one or more movements to pivot an instrument based on the captured position of the instrument H in order to align the trajectory corresponding to the portion of the motion path and the preoperative trajectory. In this example, the processing device 122 is configured to provide the one or more movements to pivot the instrument to the robotic device 140. The robotic device 140, as described herein, is configured to convert the one or more movements to pivot into instructions for enabling movement of the robotic device 140 along a determined trajectory.

In another example, the determined angle between the preoperative trajectory and the trajectory corresponding to the portion of the motion path may be compared to one or more ranges associated with one or more scores. Based on the score, a varying visual effect (e.g., a blinking color) may be displayed with the trajectory corresponding to the portion of the motion path. For example, if the angle is within a range associated with a higher likelihood of breaching a perimeter of the pedicle, then the trajectory corresponding to the portion of the motion path is displayed in a blinking red color. In another example, if the angle is within a range corresponding to a high degree of correlation to the preoperative trajectory, then the trajectory corresponding to the portion of the motion path is displayed in a green color.

In another example, the processing device 122 is configured to determine that the overlaid portion of the determined motion path intersects with one or more predetermined boundaries within the surgical site. In this example, the processing device 122 is configured to provide for display a varying visual effect of the overlaid at least a portion of the determined motion path via the display device 126.

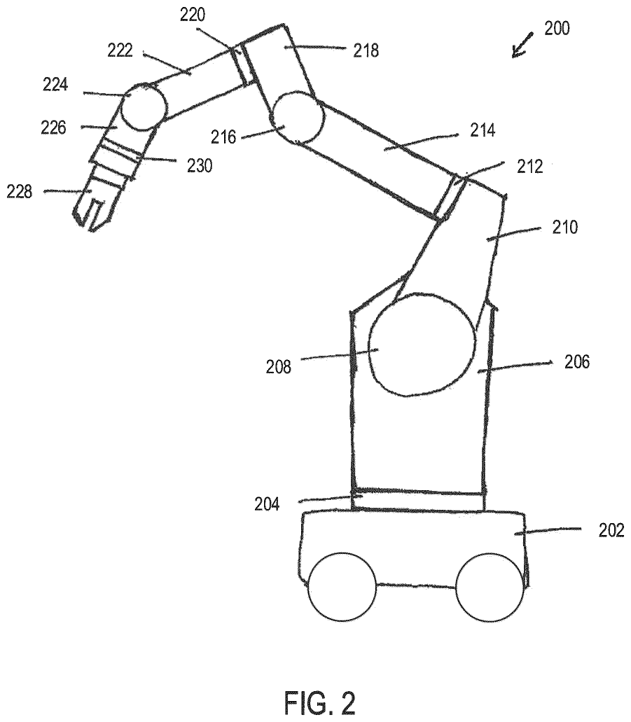

FIG. 2 illustrates an example robotic device 200 that may be used during a surgical procedure. The robotic device 200 may contain hardware, such as a processor, memory or storage, and sensors that enable the robotic device 200 for use in a surgical procedure. The robotic device 200 may be powered by various means such as electric motor, pneumatic motors, hydraulic motors, etc. The robotic device 200 includes a base 202, links 206, 210, 214, 218, 222, and 226, joints 204, 208, 212, 216, 220, 224, and 230, and manipulator 228.

The base 202 may provide a platform in order to provide support for the robotic device 200. The base 202 may be stationary or coupled to wheels in order to provide movement of the robotic device 200. The base 202 may comprise any number of materials such as aluminum, steel, stainless steel, etc., that may be suitable for a given environment associated with the robotic device 200.

The links 206, 210, 214, 218, 222, and 226 may be configured to be moved according to a programmable set of instructions. For instance, the links may be configured to follow a predetermined set of movements (e.g., a motion path corresponding to a captured pose of an instrument) in order to accomplish a task under the supervision of a user. By way of example, the links 206, 210, 214, 218, 222, and 226 may form a kinematic chain that defines relative movement of a given link of links 206, 210, 214, 218, 222, and 226 at a given joint of the joints 204, 208, 212, 216, 220, 224, and 230.

The joints 204, 208, 212, 216, 220, 224, and 230 may be configured to rotate using a mechanical gear system. In one example, the mechanical gear system is driven by a strain wave gearing, a cycloid drive, etc. The mechanical gear system selected would depend on a number of factors related to the operation of the robotic device 200 such as the length of the given link of the links 206, 210, 214, 218, 222, and 226, speed of rotation, desired gear reduction, etc. Providing power to the joints 204, 208, 212, 216, 220, 224, and 230 allows for the links 206, 210, 214, 218, 222, and 226 to be moved in a way that allows the manipulator 228 to interact with an environment.

In one example, the manipulator 228 is configured to allow the robotic device 200 to interact with the environment according to one or more constraints. In one example, the manipulator 228 performs appropriate placement of an element through various operations such as gripping a surgical instrument. By way of example, the manipulator 228 may be exchanged for another end effector that would provide the robotic device 200 with different functionality.

In one example, the robotic device 200 is configured to operate according to a robot operating system (e.g., an operating system designed for specific functions of the robot). A robot operating system may provide libraries and tools (e.g., hardware abstraction, device drivers, visualizers, message passing, package management, etc.) to enable robot applications.

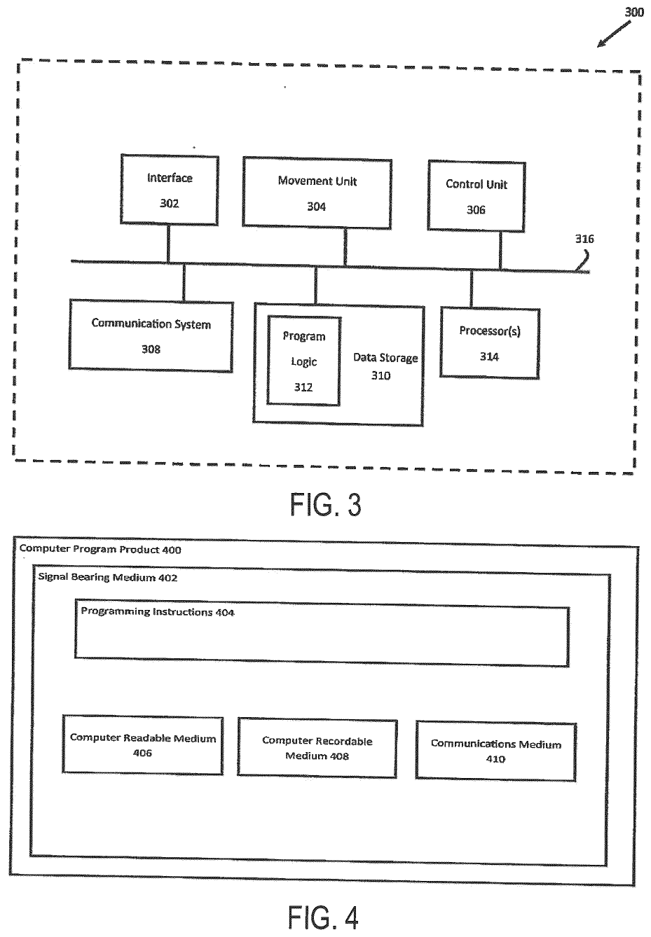

FIG. 3 is a block diagram of a computing device 300, according to an example embodiment. In some examples, some components illustrated in FIG. 3 may be distributed across multiple computing devices (e.g., desktop computers, servers, hand-held devices, etc.). However, for the sake of the example, the components are shown and described as part of one example device. The computing device 300 may include an interface 302, a movement unit 304, a control unit 306, a communication system 308, a data storage 310, and a processor 314. Components illustrated in FIG. 3 may be linked together by a communication link 316. In some examples, the computing device 300 may include hardware to enable communication within the computing device 300 and another computing device (not shown). In one embodiment, the robotic device 140 or the robotic device 200 may include the computing device 300.

The interface 302 may be configured to allow the computing device 300 to communicate with another computing device (not shown). Thus, the interface 302 may be configured to receive input data from one or more devices. In some examples, the interface 302 may also maintain and manage records of data received and sent by the computing device 300. In other examples, records of data may be maintained and managed by other components of the computing device 300. The interface 302 may also include a receiver and transmitter to receive and send data. In some examples, the interface 302 may also include a user-interface, such as a keyboard, microphone, touch screen, etc., to receive inputs as well. Further, in some examples, the interface 302 may also interface with output devices such as a display, speaker, etc.

In one example, the interface 302 may receive an input indicative of location information corresponding to one or more elements of an environment in which a robotic device (e.g., robotic device 140, robotic device 200) resides. In this example, the environment may be an operating room in a hospital comprising a robotic device configured to function during a surgical procedure. The interface 302 may also be configured to receive information associated with the robotic device. For instance, the information associated with the robotic device may include operational characteristics of the robotic device and a range of motion with one or more components (e.g., joints 204, 208, 212, 216, 220, 224, and 230) of the robotic device (e.g., robotic device 140, robotic device 200).

The control unit 306 of the computing device 300 may be configured to run control software which exchanges data with components (e.g., robot arm 141, robot pedal 142, joints 204, 208, 212, 216, 220, 224, and 230, manipulator 228, etc.) of a robotic device (e.g., robotic device 140, robotic device 200) and one or more other devices (e.g., processing device 122, tracking device 130, etc.). The control software may communicate with a user through a user interface and display monitor (e.g., display 126) in communication with the robotic device. The control software may also communicate with the tracking device 130 and the processing device 122 through a wired communication interface (e.g., parallel port, Universal Serial Bus port, etc.) and/or a wireless communication interface (e.g., antenna, transceivers, etc. configured for communication via BLUETOOTH, WIFI, other modalities, or combinations thereof). The control software may communicate with one or more sensors to measure the efforts exerted by the user at the instrument T mounted to a robot arm (e.g., robot arm 141, link 226). The control software may communicate with the robot arm to control the position of the robot arm relative to the marker 150.

As described above, the control software may be in communication with the tracking device 130. In one scenario, the tracking device 130 may be configured to track the marker 150 that is attached to the patient P. By way of example, the marker 150 may be attached to a spinous process of a vertebra of the patient P. In this example, the marker 150 may include one or more infrared reflectors that are visible to the tracking device 130 to determine the location of the marker 150. In another example, multiple markers may be attached to one or more vertebrae and used to determine the location of the instrument T.

In one example, the tracking device 130 may provide updates in near real-time of the location information of the marker 150 to the control software of the robotic device 140. The robotic device 140 may be configured to receive updates to the location information of the marker 150 from the tracking device 130 via a wired and/or wireless interface. Based on the received updates to the location information of the marker 150, the robotic device 140 may be configured to determine one or more adjustments to a first position of the instrument T in order to maintain a desired position of the instrument T relative to the patient P.

In one embodiment, the control software may include independent modules. In an exemplary embodiment, these independent modules run simultaneously under a real time environment and use a shared memory to ensure management of the various tasks of the control software. The modules may have different priorities, such as a safety module having the highest priority, for example. The safety module may monitor the status of the robotic device 140. In one scenario, the safety module may send an instruction to the control unit 306 to stop the robot arm 141 when a critical situation is detected, such as an emergency stop, software failure, or collision with an obstacle, for example.

In one example, the interface 302 is configured to allow the robotic device 140 to communicate with other devices (e.g., processing device 122, tracking device 130). Thus, the interface 302 is configured to receive input data from one or more devices. In some examples, the interface 302 may also maintain and manage records of data received and sent by other devices. In other examples, the interface 302 may use a receiver and transmitter to receive and send data.

The interface 302 may be configured to manage the communication between a user and control software through a user interface and display screen (e.g., via displays 123 and 124). The display screen may display a graphical interface that guides the user through the different modes associated with the robotic device 140. The user interface may enable the user to control movement of the robot arm 141 associated with the beginning of a surgical procedure, activate a tracking mode to be used during a surgical procedure, and stop the robot arm 141, for example.

The movement unit 304 may be configured to determine the movement associated with one or more components of the robot arm 141 to perform a given procedure. In one embodiment, the movement unit 304 may be configured to determine the trajectory of the robot arm 141 using forward and inverse kinematics. In one scenario, the movement unit 304 may access one or more software libraries to determine the trajectory of the robot arm 141. In another example, the movement unit 304 is configured to receive one or more instructions for actuating the one or more components of the robotic device 140 from the processing device 122 according to the captured motion data of an instrument based on a user-defined path.

The movement unit 304 may be configured to simulate an operation of the robotic device 140 moving an instrument T along a given path. In one example, based on the simulated operation, the movement unit 304 may determine a metric associated with the instrument T. Further, the movement unit 304 may be configured to determine a force associated with the metric according to the simulated operation. In one example, the movement unit 304 may contain instructions that determine the force based on an open kinematic chain.

The movement unit 304 may include a force module to monitor the forces and torques measured by one or more sensors coupled to the robot arm 141. In one scenario, the force module may be able to detect a collision with an obstacle and alert the safety module.

The control unit 306 may be configured to manage the functions associated with various components (e.g., robot arm 141, pedal 142, etc.) of the robotic device 140. For example, the control unit 306 may send one or more commands to maintain a desired position of the robot arm 141 relative to the marker 150. The control unit 306 may be configured to receive movement data from a movement unit 304.

In one scenario, the control unit 306 can instruct the robot arm 141 to function according to a cooperative mode. In the cooperative mode, a user is able to move the robot arm 141 manually by holding the tool T coupled to the robot arm 141 and moving the instrument T to a desired position. In one example, the robotic device 140 may include one or more force sensors coupled to an end effector of the robot arm 141. By way of example, when the user grabs the instrument T and begins to move it in a direction, the control unit 306 receives efforts measured by the force sensor and combines them with the position of the robot arm 141 to generate the movement desired by the user.

In one scenario, the control unit 306 can instruct the robot arm 141 to function according to a given mode that causes the robotic device 140 to maintain a relative position of the instrument T to a given IR reflector or emitters (e.g., the marker 150). In one example, the robotic device 140 may receive updated position information of the marker 150 from the tracking device 130 and adjust. In this example, the movement unit 304 may determine, based on the received updated position information of the marker 150, which joint(s) of the robot arm 141 should move to maintain the relative position of the instrument T with the marker 150.

In another scenario, a restrictive cooperative mode may be defined by a user to restrict movements of the robotic device 140. For the example, the control unit 306 may restrict movements of the robot arm 141 to a plane or an axis, according to user preference. In another example, the robotic device 140 may receive information pertaining to one or more predetermined boundaries within the surgical site that should not intersect with a portion of a determined motion path.

In one embodiment, the robotic device 140 may be in communication with the processing device 122. In one example, the robotic device 140 may provide the position and orientation data of the instrument T to the processing device 122. In this example, the processing device 122 may be configured to store the position and orientation data of the instrument T for further processing. In one scenario, the image-processing device 122 may use the received position and orientation data of the instrument T to overlay a virtual representation of the instrument Ton display 126.

In one embodiment, a sensor configured to detect a pressure or force may be coupled to the last joint of the robot arm (e.g., link 226). Based on a given movement of the robot arm, the sensor may provide a reading of the pressure exerted on the last joint of the robot arm to a computing device (e.g., a control unit of the robotic device). In one example, the robotic device may be configured to communicate the force or pressure data to a computing device (e.g., processing device 122). In another embodiment, the sensor may be coupled to an instrument such as a retractor. In this embodiment, the force or pressure exerted on the retractor and detected by the sensor may be provided to the robotic device (e.g., robotic device 140, robotic device 200) or a computing device (e.g., processing device 122) or both for further analysis.

In one scenario, the robotic device may access movement data stored in a memory of the robotic device to retrace a movement along a determined motion path. In one example, the robotic device may be configured to move the surgical tool along the determined motion path to reach or move away from the surgical site.

In another aspect, a robotic device (e.g., robotic device 140, robotic device 200) may assist with movement of an instrument along the determined motion path. In one scenario, the surgeon may plan for a trajectory of a pedicle screw intra-operatively by holding a portable instrument (e.g., instrument H) at a position outside the skin and seeing how the trajectory intersects the anatomy of concern by overlaying at least a portion of the determined motion path onto an image of the surgical site. In one example, the robotic device may be configured to move along a different trajectory until the tool intersects with a pedicle screw trajectory captured via the portable instrument. In this example, the robotic device may provide a signal to a computing device (e.g., processing device 122, computing device 300) which in turn could notify the surgeon via an audible or visual alert that the ideal pedicle screw trajectory has been reached.

In another scenario, once the instrument coupled to a robot arm (e.g., robot arm 141, links 206, 210, 214, 218, 222, and 226) of a robotic device reaches a desired pedicle screw trajectory, the robotic device may be configured to receive an input from the surgeon to travel along the desired pedicle screw trajectory. In one example, the surgeon may provide an input to the robotic device (e.g., depressing the pedal 142) to confirm the surgeon's decision to enable the robotic device to travel along the desired pedicle screw trajectory. In another example, a user may provide another form of input to either the robotic device or the computing device to assist with movement of an instrument along a determined motion path.

In one scenario, once the robotic device has received confirmation to travel along the desired pedicle screw trajectory, the robotic device may receive instructions from the movement unit 304 to pivot from the current trajectory to the desired pedicle screw trajectory. The movement unit 304 may provide the control unit 306 the required movement data to enable the robotic device to move along the desired pedicle screw trajectory.

In another aspect of the present invention, a robotic device (e.g., robotic device 140, robotic device 200) may be configured to pivot about an area of significance based on the captured pose of a portable instrument (e.g., instrument H). For example, the robotic device may be configured to pivot a retractor about the tip of the retractor so that all steps associated with retraction of soft tissue need not be repeated. In one example, the movement unit 304 may determine the trajectory required to pivot the retractor.

In one example, the robotic device may be coupled to a retractor that is holding soft tissue away from a surgical site. In this example, a surgeon want to slightly reposition the retractor due to a patient movement. To do so, the surgeon may activate a mode on the robotic device that causes the retractor to pivot by moving the robot arm (e.g., robot arm 141, links 206, 210, 214, 218, 222, and 226) according to a trajectory determined by the movement unit 304. In one example, a user may input the direction and amount of movement desired via a computing device (e.g., the processing device 122, computing device 300). After the direction and amount of movement have been entered, the user (e.g., a surgeon) may interface with the robotic device (e.g., depress the pedal 142) to begin the movement of the instrument coupled to the robot arm. In one example, the robotic device may allow a user to view a different aspect of the anatomy without disengaging from a docking point.

In another example, the movement unit 304 may provide one or more trajectories based on the captured pose of the portable instrument (e.g., instrument H) to a computing device (e.g., processing device 122) for display on display 126. In this example, a user may choose from one or more predetermined movements associated with a given procedure. For example, a given predetermined movement may be associated with a specific direction and amount of movement to be performed using depressing the pedal 142 of the robotic device 140.

In another aspect of the present invention, one or more infrared (IR) reflectors or emitters may be coupled to a robot arm (e.g., robot arm 141, links 206, 210, 214, 218, 222, and 226) of the robotic device (e.g., robotic device 140, robotic device 200). In one scenario, the tracking device 130 may be configured to determine the location of the one or more IR reflectors or emitters prior to beginning operation of the robotic device. In this scenario, the tracking device 130 may provide the location information of the one or more IR reflectors or emitters to a computing device (e.g., processing device 122, computing device 300) for further processing.

In one example, the processing device 122 or computing device 300 may be configured to compare the location information of the one or more IR reflectors or emitters coupled to the robot arm with data stored on a local or remote database that contains information about the robotic device (e.g., a geometric model of the robotic device) to assist in determining a location or position of the robot arm. In one example, the processing device 122 may determine a first position of the robot arm from information provided by the tracking device 130. In this example, the processing device 122 may provide the determined first position of the robot arm to the robotic device or a computing device (e.g., computing device 300). In one example, the robotic device may use the received first position data to perform a calibration of one or more elements (e.g., encoders, actuators) associated with the one or more joints of the robot arm.

In one scenario, an instrument coupled to the robot arm of the robotic device may be used to determine a difference between an expected tip location of the instrument and the actual tip location of the instrument. In this scenario, the robotic device may proceed to move the instrument to a known location by the tracking device 130 so that the tip of the tool is in contact with the known location. The tracking device 130 may capture the location information corresponding to the one or more IR reflectors or emitters coupled to the robot arm and provide that information to the robotic device or a computing device (e.g., processing device 122, computing device 300). Further, either the robotic device or the computing device may be configured to adjust a coordinate system offset between the robotic device and the tracking device 130 based on the an expected tip location of the tool and the actual tip location of the tool.

In another aspect, a force or pressure sensor may be coupled to a robot arm (e.g., robot arm 141, links 206, 210, 214, 218, 222, and 226) of a robotic device (e.g., robotic device 140, robotic device 200). In one example, the force or pressure sensor may be located on an end effector of the robot arm. In another example, the force or pressure sensor may be coupled to a given joint of the robotic arm. The force or pressure sensor may be configured to determine when a force or pressure reading is above a resting threshold. The resting threshold may be based on a force or pressure experienced at the sensor when the end effector is holding the instrument without any additional forces or pressure applied to the instrument (e.g., a user attempting to move the instrument). In one example, the robot arm may stop moving if the force or pressure reading is at or below the resting threshold.

In one example, the movement of the robot arm 141 may be controlled by depression of the pedal 142. For example, while the pedal 142 is depressed, the control unit 306 and the movement unit 304 may be configured to receive any measures of force or pressure from the one or more force sensors and used the received information to determine the trajectory of the robot arm 141.

In another example, the movement of the robot arm 141 may be regulated by how much the pedal 142 is depressed. For example, if the user depresses the pedal 142 to the full amount, the robot arm 141 may move with a higher speed compared to when the pedal 142 is depressed at half the amount. In another example, the movement of the robot arm 141 may be controlled by a user interface located on the robotic device.

In one example, the robotic device (e.g., robotic device 140, robotic device 200) may be configured to store, in a local or remote memory, movement data that corresponds to a user defined motion path based on movement of a portable instrument. In this example, the robotic device may be configured to travel in one or more directions along a trajectory corresponding to the stored movement data. For example, a surgeon may instruct the robotic device to reverse along the trajectory corresponding to the stored movement data.

In another aspect of the present invention, a robotic device (e.g., robotic device 140, robotic device 200) may be used to navigate one or more surgical instruments and provide the navigation information to a computing device (e.g., processing device 122, computing device 300) for further processing. In one example, the computing device may be configured to determine a virtual representation of the surgical instrument. Further, the computing device may be configured to overlay the virtual representation of the surgical instrument on a two-dimensional or three-dimensional image of the surgical site.

In one example, the robotic device may perform a calibration procedure between the tracking device 130 in order to remove the dependence on the tracking device 130 for location information in the event that a line of sight between the robotic device and the tracking device 130 is blocked. In one example, using a robotic device that has been registered to a navigation system, as described herein, and a patient's three-dimensional image that corresponds to the surgical site may allow the robotic device to become independent of the degradation of accuracy with distance associated with the tracking device 130.

The communication system 308 may include a wired communication interface (e.g., parallel port, USB, etc.) and/or a wireless communication interface (e.g., antenna, transceivers, etc.) to receive and/or provide signals from/to external devices. In some examples, the communication system 308 may receive instructions for operation of the processing device 122. Additionally or alternatively, in some examples, the communication system 308 may provide output data.

The data storage 310 may store program logic 312 that can be accessed and executed by the processor(s) 314. The program logic 312 may contain instructions that provide control to one or more components of the processing device 122, the robotic device 140, the robotic device 200, etc. For example, program logic 312 may provide instructions that adjust the operation of the robotic device 200 based one on or more user-defined trajectories associated with a portable instrument. The data storage 310 may comprise one or more volatile and/or one or more non-volatile storage components, such as optical, magnetic, and/or organic storage, and the data storage may be integrated in whole or in part with the processor(s) 314.

The processor(s) 314 may comprise one or more general-purpose processors and/or one or more special-purpose processors. To the extent the processor 314 includes more than one processor, such processors may work separately or in combination. For example, a first processor may be configured to operate the movement unit 304, and a second processor of the processors 314 may operate the control unit 306.

Still further, while each of the components are shown to be integrated in the processing device 122, robotic device 140, or robotic device 200, in some embodiments, one or more components may be removably mounted to otherwise connected (e.g., mechanically or electrically) to the processing device 122, robotic device 140, or robotic device 200 using wired or wireless connections.

FIG. 4 depicts an example computer readable medium configured according to an example embodiment. In example embodiments, an example system may include one or more processors, one or more forms of memory, one or more input devices/interfaces, one or more output devices/interfaces, and machine readable instructions that when executed by the one or more processors cause the system to carry out the various functions tasks, capabilities, etc., described above.

As noted above, in some embodiments, the disclosed techniques (e.g., functions of the robotic device 140, robotic device 200, processing device 122, computing device 300, etc.) may be implemented by computer program instructions encoded on a computer readable storage media in a machine-readable format, or on other media or articles of manufacture. FIG. 4 is a schematic illustrating a conceptual partial view of an example computer program product that includes a computer program for executing a computer process on a computing device, arranged according to at least some embodiments disclosed herein.

In one embodiment, an example computer program product 400 is provided using a signal bearing medium 402. The signal bearing medium 402 may include one or more programming instructions 404 that, when executed by one or more processors, may provide functionality or portions of the functionality described above with respect to FIGS. 1-3. In some examples, the signal bearing medium 402 may be a computer-readable medium 406, such as, but not limited to, a hard disk drive, a Compact Disc (CD), a Digital Video Disk (DVD), a digital tape, memory, etc. In some implementations, the signal bearing medium 402 may be a computer recordable medium 408, such as, but not limited to, memory, read/write (R/W) CDs. R/W DVDs, etc. In some implementations, the signal bearing medium 402 may be a communication medium 410 (e.g., a fiber optic cable, a waveguide, a wired communications link, etc.). Thus, for example, the signal bearing medium 402 may be conveyed by a wireless form of the communications medium 410.

The one or more programming instructions 404 may be, for example, computer executable and/or logic implemented instructions. In some examples, a computing device may be configured to provide various operations, functions, or actions in response to the programming instructions 404 conveyed to the computing device by one or more of the computer readable medium 406, the computer recordable medium 408, and/or the communications medium 410. One or more of the software examples and/or method operations described herein can be implemented as instructions and stored as part of the programming instructions 404 on the signal bearing medium 402.

The computer readable medium 406 may also be distributed among multiple data storage elements, which could be remotely located from each other. The computing device that executes some or all of the stored instructions could be an external computer, or a mobile computing platform, such as a smartphone, tablet device, personal computer, wearable device, etc. Alternatively, the computing device that executes some or all of the stored instructions could be remotely located computer system, such as a server.

An additional robotic surgical platform that can benefit from examples disclosed herein is described in U.S. Pat. No. 11,135,015, filed Jul. 17, 2018, as application Ser. No. 16/037,175.

An additional robotic surgical platform that can benefit from examples disclosed herein is described in U.S. patent application Ser. No. 17/696,203, which was filed Mar. 16, 2022.

Examples described herein include improvements to a distal section of a robot arm. The distal section described herein can be implemented into the robot arm itself and/or be configured as an end effector configured for attachment to a distal link of the robot arm.

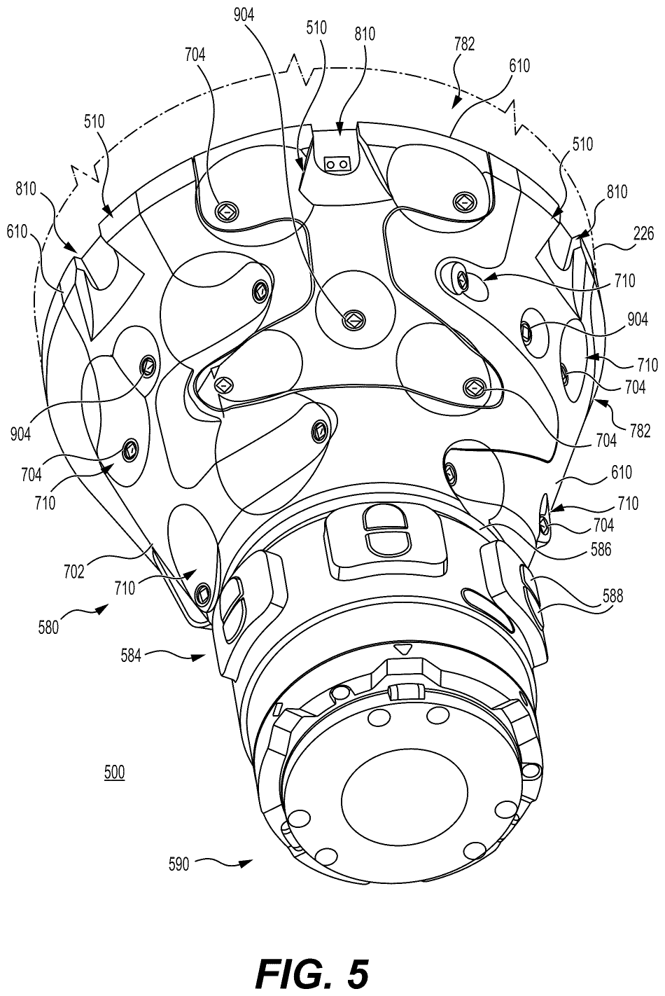

FIG. 5 illustrates a distal section of a robot arm (or an end effector coupled to a distal section of a robot arm) 500 having a tracking section 580, a user interface section 584, and a tool connector section 590. The distal section can be connected to a link 226 of the robot arm 500 that connects to a base 202 via one or more additional links, joints, and other components as described elsewhere herein. In an example, one or more of the tracking section 580, user interface section 584, and tool connector section 590 are non-sterile and are covered by a drape to separate them from a sterile operating area. A drape can be disposed between the tool connector section 590 and a tool connected thereto. In an example, the components proximal to the drape are not configured to be sterilized (e.g., one or more of the components would more likely than not be harmed by a sterilization process).

The tool connector section 590 is a section of the arm 500 configured to couple with a tool (e.g., an end effector). Example tools include guide tubes, retractor mounts, cutting tools, other tools, or combinations thereof.

The user interface section 584 includes a display 586 and buttons 588. The display 586 and buttons 588 can be used to control one or more aspects of the robot or an associated system (e.g., a navigation system that tracks the robot and/or a surgical computer system that has features designed to assist in a surgery). In an example, the display 586 is a light emitting ring configured to change colors and light up at different intensities or patterns of intensities to communicate information to a user. In addition or instead, the display 586 can define a plurality of pixels to convey additional information. The buttons 588 can be one or more elements by which the robot can receive information from a user.

The tracking section 580 is a section of the robot arm 500 configured to facilitate tracking the robot arm 500 with a navigation system (e.g., tracking device 130). As illustrated, the tracking section 580 has the shape of a truncated cone (e.g., a frustoconical shape with the smaller diameter end being distal to the larger diameter end). The tracking section can be broken into different faces 510.

The faces 510 are logically or physically distinct regions of the tracking section 580 that are separably trackable by the navigation system. Having multiple faces 510 can facilitate the tracking of the robot arm 500 as it is rotated or otherwise moved during use. For instance, there can be a predetermined association between each face 510 of the robot arm 500 and the tool connector section 590 (e.g., a known three dimensional vector points from the center of a particular face to a center of the tool connector section 59). Such a predetermined association can be combined with other predetermined associations such that knowledge of the location of a particular face 510 in space can be used to determine the location of another portion of the robot arm 500 or even a tool connected to the robot arm 500 (e.g., a guide tube end effector). Thus, visibility of any one face 510 to the navigation system can be used to track a point of interest associated with the face 510.

In some implementations, a single face 510 may be sufficient. In other examples, multiple faces 510 may be used to improve tracking. For instance, where the distal portion of the robot arm 500 is able to roll, such motion may cause a face 510 to be oriented away from a tracking camera or otherwise be obscured or difficult to track. Multiple faces 510 can be used such that even if one or more faces 510 are obscured, there is a sufficiently high likelihood that one of the faces 510 will be usefully visible to a tracking system. The angling of the tracking section 580 (e.g., because of the frustoconical shape) can facilitating tracking of the faces 510 by increasing orthogonality of the normals of the faces 510 and to the navigation camera's orientation.

Each face 510 be associated with a set of one or more fiducials 704 (typically at least four fiducials 704) arranged as a fiducial array 782. The fiducial array 782 can be predetermined arrangement of a set of fiducials 704. The fiducial array 782 can then be tracked by the navigation system to determine the position and orientation of an associated face 510 (e.g., in six degrees of freedom).

The fiducials 704 of a fiducial array 782 may but need not remain only on their associated face 510. In some instances, fiducials 704 associated with one face 510 can actually be disposed on or within the boundaries of another face 510. In addition or instead, the faces 510 can have a convex boundary. Bounding boxes of the faces 510 or fiducial arrays 782 may nonetheless overlap in certain configurations.

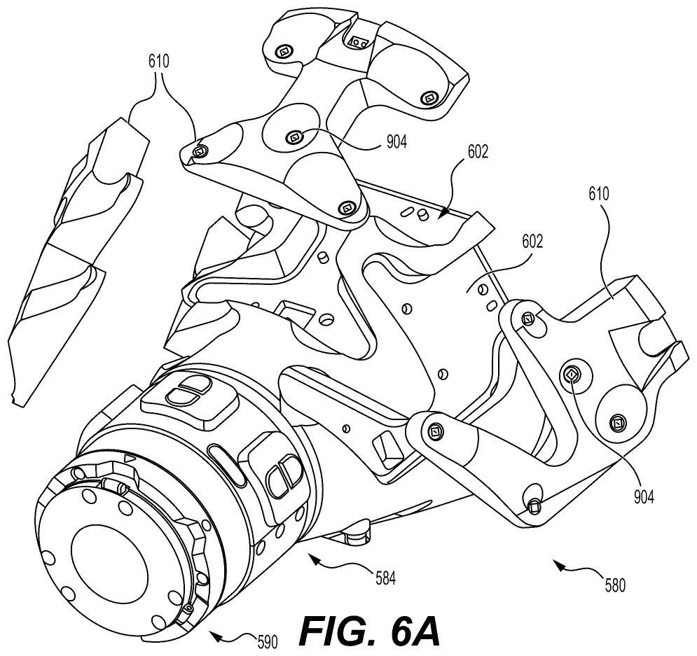

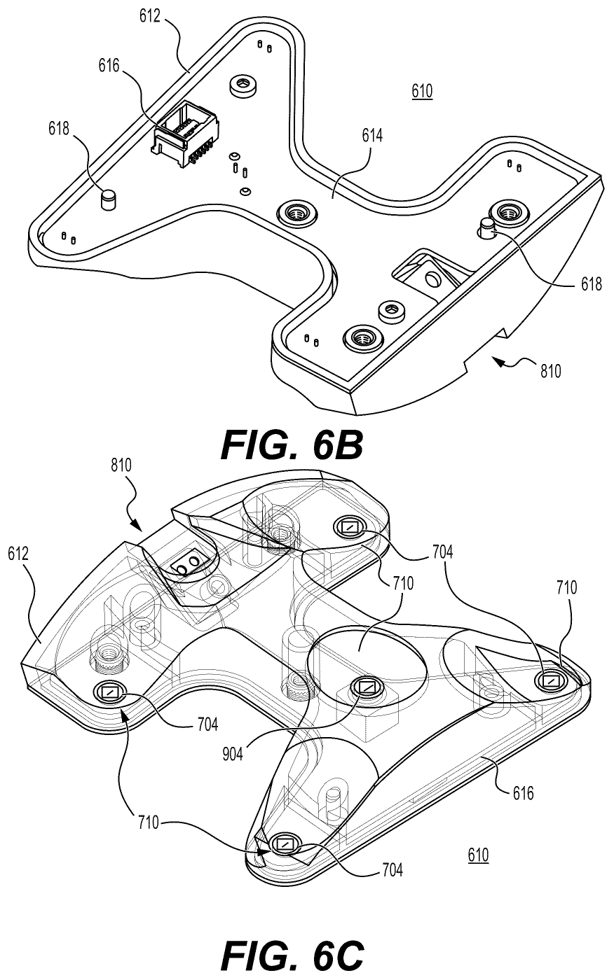

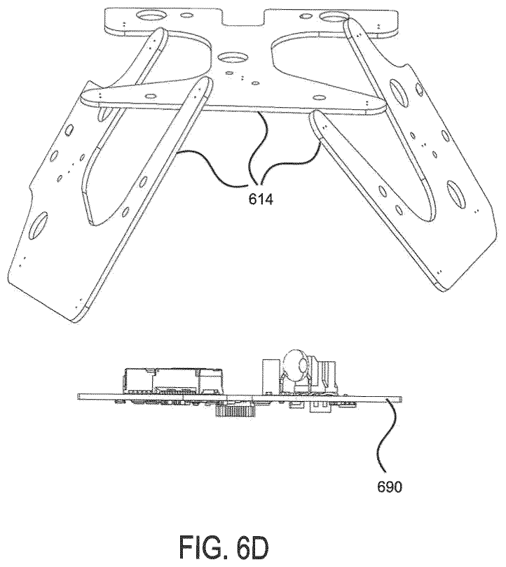

As described in more detail in association with FIGS. 6A-C, robot arm 500 can include pods 610 having the fiducials 704.

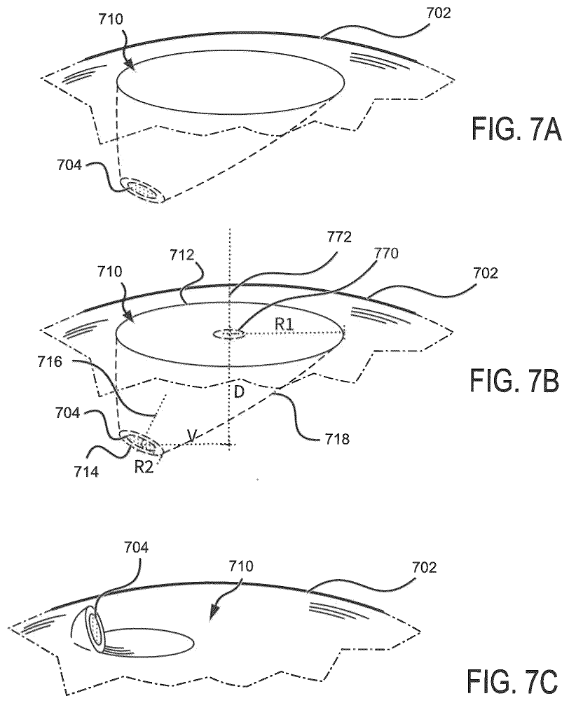

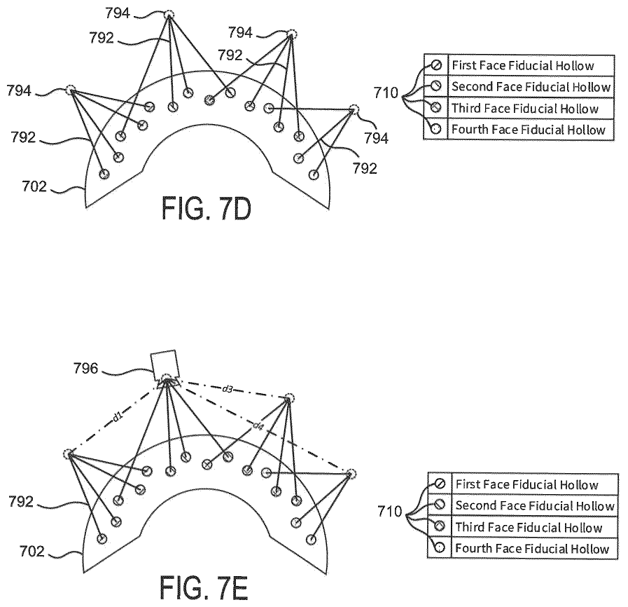

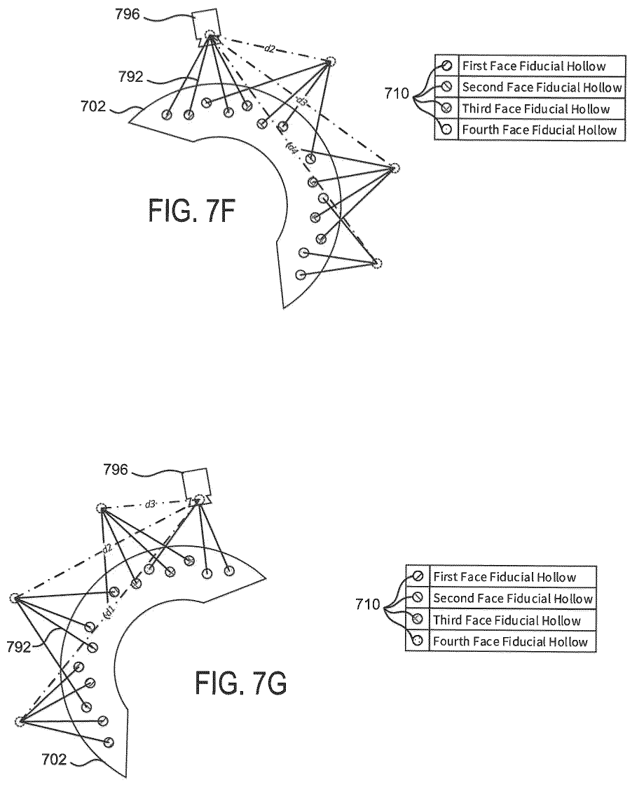

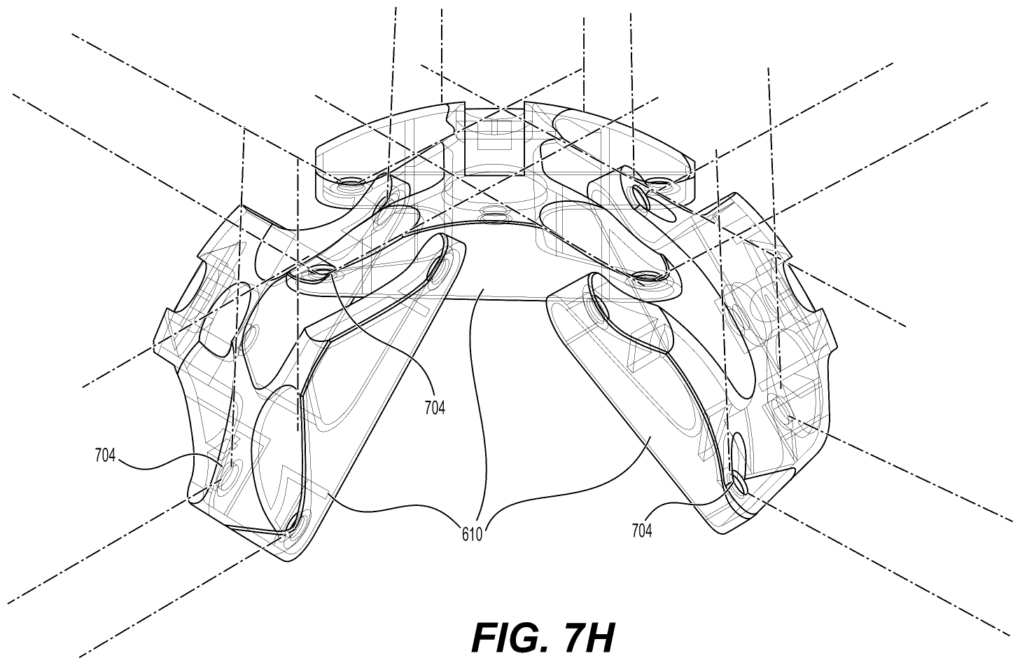

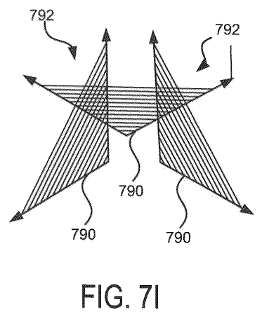

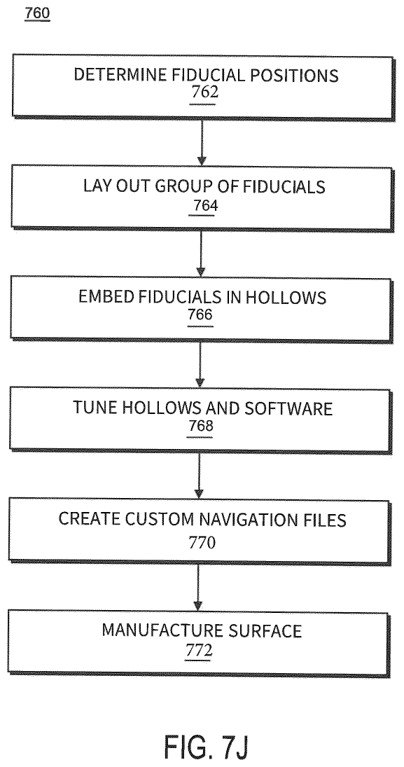

As described in association with FIGS. 7A-H, the fiducials 704 can be disposed within hollows 710.

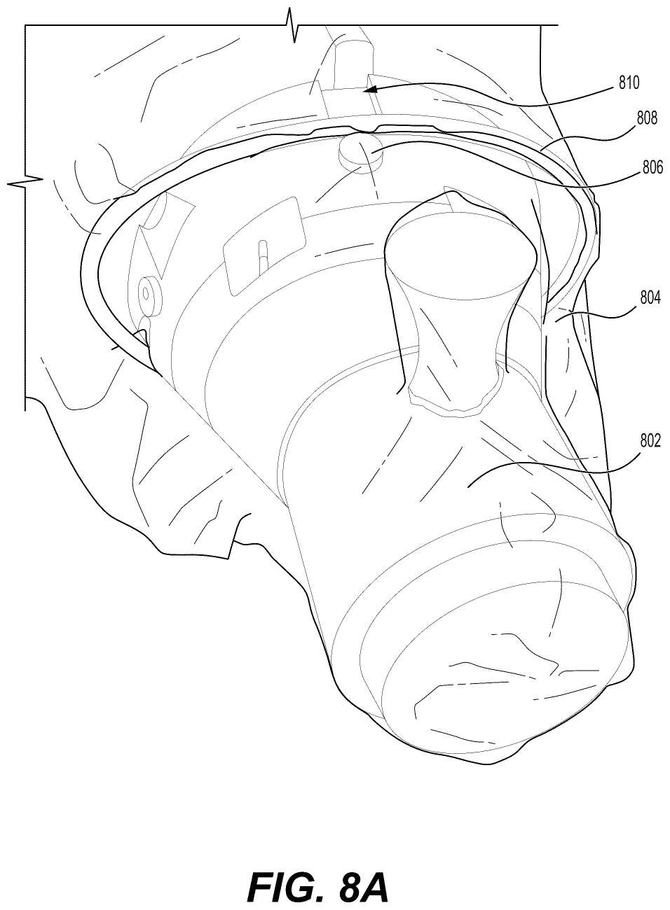

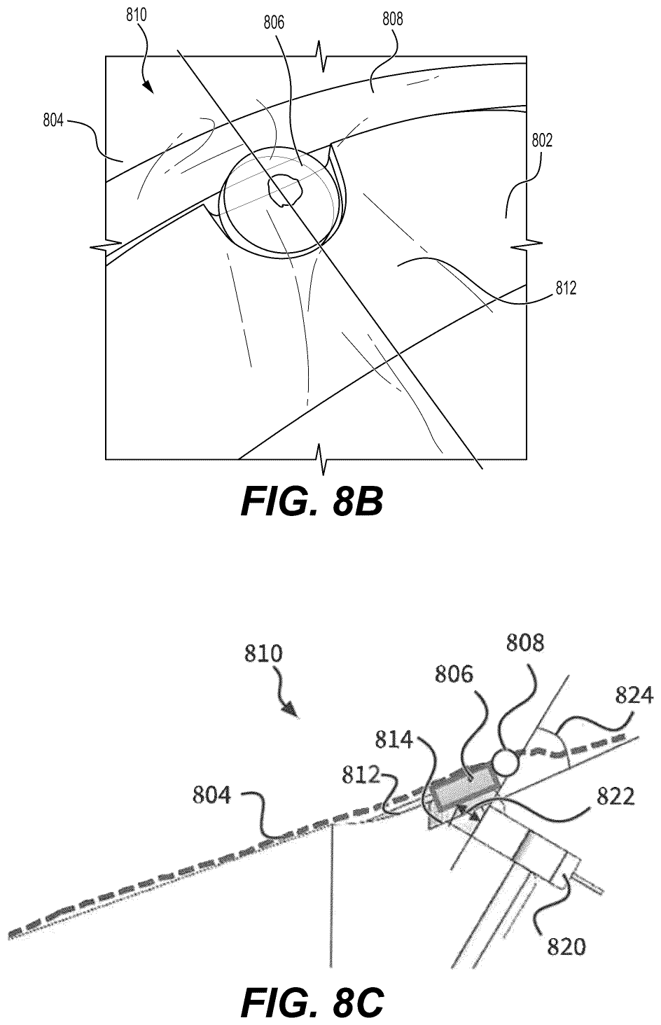

As described in association with FIGS. 8A-C, the robot arm 500 includes capture areas 810 configured to retain a drape.

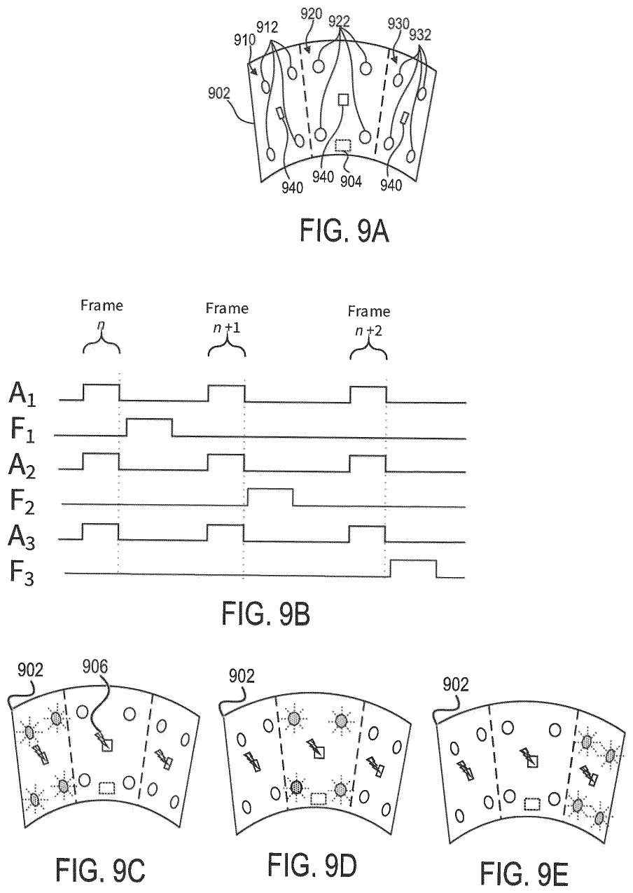

As described in association with FIGS. 9A-E, the pods 610 (or another portion of the robot arm 500) can include one or more activation sensors 940.

FIGS. 6A-C illustrate pods 610 of a robot arm 500 having fiducials 704 disposed within hollows 710. In an example, each pod 610 can be a self-contained fiducial 704 or set of fiducials 704 that form a face 510 or fiducial array 782. Packaging or associating each face 510 or fiducial array 782 in its own pod 610 can increase fiducial-to-fiducial and face-to-face accuracy as well as making the robot arm 500 easier to manufacture. As illustrated, the robot arm 500 can include receptacles 602 configured to receive the pods 610.

The pods 610 can include a housing 612 in which one or more components are disposed. As illustrated, such components can include a circuit board 614, a connector 616, guide pins 618, fiducials 704, sensors, and other components. The pods 610 can further include capture areas 810 configured to retain a drape. For example, the housing 612 can define the capture areas 810 such as is described in more detail in relation to FIGS. 8A-C. The housing 612 can further define the hollows 710.

The circuit board 614 can be a printed circuit board or another electronics assembly on which one or more electrical components can be disposed or mounted. The circuit board 614 can be a stiff board or can be flexible. Example electrical components include fiducials 704 in the form of active fiducials (e.g., infrared emitting diodes), the connector 616, a sensor 820 (e.g., a drape detection sensor described elsewhere herein or an activation sensor described elsewhere herein), other components, or combinations thereof. In the illustrated example, there is a single circuit board 614 per pod 610.

Mounting the fiducials 704 to a single printed circuit board 614 increases the accuracy of the system due to the precise positioning of the fiducials 704 achievable by the printed circuit board manufacturing process. In an example, the fiducials 704 are mounted flat to a surface of the circuit board 614 (e.g., conforming the surface of the circuit board 614). In an example, the fiducials 704 are mounted oblique to or otherwise angled relative to the surface of the circuit board 614. In an example, all of the fiducials 704 of a pod 610 are mounted to a same circuit board 614. In an example, all of the fiducials 704 of a pod 610 are mounted to a same circuit board 614 with the normals of each fiducial 704 being oriented in a same direction.

In other examples, there can be more than one circuit board 614 per pod 610 that may be electrically connected to each other. In an example, the multiple circuit boards 614 or one or more flexible circuit boards 614 can be used to orient particular components in particular directions.

The connector 616 can be an electrical connector configured to electrically connect the circuit board 614 with an electronic component of the robot arm 500 (e.g., other pods 610 or an end effector circuit board 690 as discussed below). The connector 616 can be configured to receive one or more wires or cables. In addition or instead, the pods 610 can include wireless communication or power transmission components.

The guide pins 618 can be components within the receptacles 602 facilitate accurate placement of the pod 610 within the receptacles 602. The pods 610 can include or more receivers configured to receive the one or more guide pins 618. In other examples, the pods 610 can include the one or more guide pins 618 and the receptacles 602 include one or more regions configured to receive the guide pins 618.

In examples, the pods 610 can include computing components (and one or more processors, memories, sensors, and interfaces). In some examples, the pods 610 can include a battery to power one or more electric components. In other examples, the pods 610 can be passive (e.g., lack active electronic components and use passive reflective fiducials 704). In some examples, the pods 610 include one or more other components for tracking or navigation. The pods 610 can include one or more cameras, infrared projectors, radiofrequency trackers, other components, or combinations thereof.

FIG. 6D illustrates a front view of an end effector having an end effector circuit board 690 and circuit boards 614 of three pods 610 installed in the end effector with all other components removed for ease of viewing the relative location of the circuit boards 614, 690. As illustrated, in the front view of the end effector (e.g., along a long axis of the end effector), the circuit boards 614 are oblique relative to each other. For example, the circuit boards 614 are angled by approximately 120 degrees relative to each other. In an example, the circuit boards 614 have the same angle relative to each other. In another example, the circuit boards 614 are each at different angles relative to each other. For example, the angle between a first and second circuit board 614 is less than 120 degrees (e.g., 118 degrees), the angle between the second and third circuit board 614 is more than 120 degrees (e.g., 130 degrees), and the angle between the first and third circuit boards 614 is less than 70 degrees (e.g., approximately 67 degrees). In addition, one or more of the circuit boards can be oblique relative to the end effector circuit board 690.

As can be further seen in this view, the circuit boards 614 each have a different concave shape (e.g., a shape having at least one concavity). The circuit boards 614 can be mounted such that the circuit boards 614 have intersecting bounding boxes.

The end effector circuit board 690 can provide one or more features of the end effector. In an example, the end effector circuit board 690 can provide power and data to and receive data from the circuit boards 614 of the pods 610. In an example, each respective circuit board 614 can have only a single cable (e.g., a ribbon cable or a round cable) running from the circuit board 614 to the end effector circuit board 690. The cable can include one or more wires for carrying power, data, or other uses. Having only the single cable can improve the ease of wiring the inside of the end effector and reduce the overall size. In the illustrated example, the circuit board 690 is non-circular and is perpendicular to (or at least non-parallel with) the connector for the end effector to couple to a link of the robot arm (or a connector for a tool guide). The circuit board 690 can define a plane that is disposed parallel to a length of the end effector.

Face switching refers to the behavior of switching among multiple faces 510 for tracking purposes. For instance, the switching can be from a first face 510 to a second face 510 to increase tracking accuracy because the second face 510 is more orthogonal to the camera's orientation. Switching can extend a viewing angle. But having multiple faces 510 introduces complexity. Some challenges that exist are: determining an appropriate switching threshold of when to switch to a different face 510, fiducial 704 spacing between faces, accuracy discontinuity when switching between faces 510, and extra footprint required for multiple faces 710.

Examples disclosed herein are relevant to addressing the disadvantages and providing new advantages. To improve multi-face performance, a balanced approach that reduces face switching, packs the faces 510 tightly, and provides more consistent accuracy between face 510 is the beneficial for developing a well performing multi-face system.

The design herein can be used to accomplish this feat using the following example architecture construction method: determine the minimum fiducial spacing experimentally, lay-out the faces 710 to have similar focal points, embed the individual fiducials 704 in hollows to artificially constrain visibility to resist multi-face visibility at high angles, tune the hollows and navigation software (e.g., camera-to-face angle) limits so face switching occurs before a mechanical occlusion occurs, measure the fiducial center optically, and create custom navigation files to reduce optical fitting errors by the localization system.

FIG. 7A illustrates a surface 702 having a fiducial 704 disposed relative to a hollow 710 in the surface 702.

The surface 702 is a surface of an object to be tracked, such as the surface of the tracking section 580 of the robot arm 500. The illustrated examples primarily relate to a surface of a robotic arm that is tracked with a surgical navigation system to facilitate a medical procedure. But the examples described herein can be applied in other contexts. In some examples, the surface 702 is a surface of a housing 612 of a pod 610.

The fiducial 704 is a trackable object used to facilitate surgical navigation. The fiducial 704 can be active or passive. In an example, the fiducial 704 is a passive infrared reflector (e.g., a retroreflector) or an active infrared emitter. In other examples, the fiducial 704 can take other forms.

The hollow 710 is a concavity of the surface 702. In many examples, the hollow 710 is presented generally as a truncated cone herein but can take other forms, such as a truncated pyramid, a frustrum, a funnel, a depression, a crater shape, an organic shape, other shapes, or combinations thereof. The hollow 710 can have any of a variety of properties or geometries as are described in more detail in FIG. 7C.

FIG. 7B annotates the surface 702, fiducial 704 and hollow 710 of FIG. 7A. While a person of ordinary skill in the art will understand a variety of ways to describe this arrangement of components, this figure describes the components relative to an imaginary fiducial 770.

The imaginary fiducial 770 is a virtual fiducial that is flush with and conforms to the surface 702. The imaginary fiducial 770 can be a reference location relative to which the actual location of the fiducial and characteristics of the hollow 710 can be described. An imaginary fiducial 770 can be thought of as the location that the fiducial 704 would occupy if the fiducial 704 were not disposed in the hollow 710 (e.g., and not customized according to this disclosure). In other examples, the imaginary fiducial 770 can be used as an arbitrary reference point on the surface 702 relative to which the fiducial 704 is described. An imaginary fiducial normal 772 extends from the imaginary fiducial 770.

The hollow 710 can define an upper boundary 712 and a lower boundary 714 between which one or more walls 718 extend. The upper boundary 712 can be boundary at which the geometry of the hollow 710 begins to deviate from the geometry of the surface 702 at the surface of the surface 702. The lower boundary 714 can be a bottom of the hollow 710. In the illustrated example, the hollow 710 generally has the shape of a truncated cone and the lower boundary 714 is the surface that cuts the cone. The lower boundary 714 can be the portion of the hollow 710 on which the fiducial 704 is disposed. A lower boundary normal 716 can extend from the lower boundary 714. As illustrated, the upper boundary 712 and the lower boundary 714 have radii R1 and R2, respectively. In other examples, the boundaries 712, 714 are not circles (e.g., they may be ellipses, or n-gons) and can instead be described by other measurements.

In this illustrated example, the hollow 710 generally has the shape of a truncated cone and so has relatively clearly defined separate lower boundary 714 and wall 718 sections. But other forms of the hollow 710 may lack such clear separation and may instead be more readily described as only having a lower boundary 714 (e.g., where the hollow 710 is bowl shaped) or only having walls (e.g., having the geometry of a pyramid terminating at a point).

In an example, the location of the fiducial 704 can be described in a coordinate space where the imaginary fiducial 770 is the origin. The rotation of the fiducial 704 can be described in reference to the imaginary fiducial normal 772. The hollow 710 can be so constructed such that the fiducial 704 is depth D lower than the imaginary fiducial 770. The offset of the fiducial 704 relative to the imaginary fiducial 770 in two-dimensions (i.e., excluding depth) can be described by two-dimensional vector V.

FIG. 7C illustrates an example of a shallow hollow 710 in which the fiducial 704 is only partially sunk beneath the surface 702. The fiducial 704 in this instance is directional and faces the hollow 710.

The hollows 710 can be specifically configured to decrease the visibility of a contained fiducial 704. In an example, the hollows 710 are configured to reduce the field of view cone of a contained fiducial 704 to a particular angle (e.g., 60 degrees from a normal of the fiducial 704). Groups of hollows 710 can cooperate to form or help define specific groups of fiducials 704. In an example, a group of fiducials 704 is configured (e.g., via their respective hollows 710) such that desired separation between fiducials 704 of different faces 510 is achieved. For instance, the desired separation can be such that a navigation camera system is more likely to detect only one group of fiducials 704 of the robot arm 500 at a time and less likely to experience unwanted face switching.

A first example set of groupings is described in more detail in relation to FIGS. 7D-G.

FIG. 7D illustrates a surface having a plurality of hollows 710 with each hollow 710 having a fiducial 704 disposed therein. Each fiducial 710 can be associated with or define a fiducial normal 792 extending therefrom. The hollows 710 can be grouped into sets associated with faces 510. The hollows 710 of each face 510 or other grouping are configured (e.g., by modifying the parameters of the hollows 710 described in FIG. 7B) such that the fiducial normals 710 converge at or toward a respective different focal point 794 from the other faces. This example illustration is in two dimensions, so the fiducial normals 792 converge at a focal point 794 so long as the normals 792 are not parallel. In three-dimensional uses, the normals 792 likely do not all truly intersect at a single focal point 794. In such instances, the focal point 794 can be understood as a location of greatest intensity for the fiducials 792. In an example, the focal point 794 of a group of fiducials 792 is a point in three-dimensional space having the minimum distance to all fiducial normals 792 of the group.

As illustrated, the fiducials 704 of a given face 510 are generally grouped close together, but they need not be entirely separate from fiducials 704 of other groups. For instance, as illustrated, a fiducial 704 of the second group is mixed within the second group of fiducials 704. As can be seen, even though the fiducials 704 of different faces are intermixed, the nature of the hollows 710 are such that the fiducials 704 of the same face 510 converge at or toward a same focal point 794. Bounding boxes of sets of the fiducials 704 of different groups can overlap.

FIG. 7E illustrates a camera 796 of a navigation system being used to track the locations of the fiducials 704 of the hollows 710 of the surface 702 of FIG. 7D and the second face is oriented towards the camera 796. As illustrated, the fiducials 704 of the second face fiducial hollows 710 are facing the camera 796 and the fiducials of the other face fiducial hollows 710 are facing away from the camera. This increases the likelihood of the camera 796 detecting the fiducials 704 of the second face 510 and less likely to detect the fiducials 704 of the other faces 510. This increases accuracy of navigation and decreases the likelihood of unwanted face switching.

In the illustrated example, the sensor of the camera 796 is disposed at the focal point 794 of the second face 510. However, the camera 796 need not be so disposed. In some examples, the hollows 710 are configured such that the focal point 794 is approximately where the camera 796 would be in an operating room to detect that face 510. The focal point 794 having the least distance from the camera 796 can be the face that is detected. In this illustrated example, the focal point 794 of the second face fiducial hollows 710 is the closest to the camera 796.

FIG. 7F illustrates a camera 796 being used to track the locations of the fiducials 704 of the hollows 710 of the surface 702 of FIG. 7D and the first face 510 is oriented towards the camera 796. In an example, the orientation changes because the camera 796 moved or because the surface 702 moved (e.g., rotated).

FIG. 7G illustrates a camera 796 being used to track the locations of the fiducials 704 of the hollows 710 of the surface 702 of FIG. 7D and the fourth face 510 is oriented towards the camera 796. In an example, the orientation changes because the camera 796 moved or because the surface 702 moved (e.g., rotated).

A second example set of groupings is described in more detail in relation to FIGS. 7H and 7I.

FIG. 7H illustrates a partial transparency view of the housings 612 of three pods 610 with lines showing cones of visibility for each fiducial 704 limited by their respective hollow 710. The fiducials 704 are labeled with A, B, or C with each letter corresponding to one of the three pods 610. Here, the hollows 710 are configured to provide a specific cone of visibility for each of their fiducials 704. In some examples, the cones of visibility provided by each of the hollows 710 of a pod 610 are the same. In some examples, the cones of visibility provided by at least one of the hollows 710 of a pod 610 is different from others of the same pod 610. As illustrated, the hollows 710 of each pod 610 are configured to constrain visibility to a predetermined cone of visibility in a sufficiently far away camera can view each of the fiducials 704 of the pod 610. In some examples, overall cone of visibility of each pod 610 is the same but oriented in different ways based on the placement of the pods 610. In some examples, the cones of visibility provided by at least one pod 610 is different from that of at least one other pod 610.

FIG. 7I illustrates a simplified view of the cones of visibility 790 for each of the pods 610 of FIG. 7H. As can be seen here, there exist overlap zones 792 in the cones of visibility 790 of adjacent pods 610. While a camera 796 is disposed within an overlap zone 792, both faces are visible. Face switching can occur while the camera 796 is so disposed.

FIG. 7J illustrates a method 760 for creating a surface 702 having hollows 796.

Operation 762 includes determining fiducial 704 positioning. This can be performed by experimenting. The positions of the fiducials 704 can be influenced by a fiducial minimum distance. The fiducial minimum distance can be a minimum distance between fiducials where the fiducials are still identifiable as separate fiducials by one or more navigation systems. In some examples, the minimum distance is specified by a navigation system's camera manufacture. In some examples, the minimum spacing can be determined experimentally by bringing two fiducials towards each other and determining the minimum distance apart the fiducials can be while still being detectable as two separate fiducials by the navigation system. A predetermined safety buffer amount can be added to the minimum value. The fiducial positioning can also be determined by defining fiducial array shapes. The shapes can be configured to be separately identifiable by the navigation system.

Operation 764 includes laying-out groups of fiducials 704 (e.g., groups can be associated with faces 510 of the surface 702) on the surface 702. In an example, this can include placing one or more polygons relative to a representation of the surface 702 where each polygon represents a group of fiducials 704 designating a face 510 (e.g., the group being a fiducial array or a pod for tracking a face) with each vertex of each respective polygon corresponding to a fiducial 704 of that group. The polygons can be shifted around until there is sufficient minimum spacing between the fiducials 704 (e.g., as defined in operation 762). In an example, non-adjacent faces can be permitted to break the minimum spacing rule if hollows can be used to physically occlude the adjacent fiducials from being usefully seen at the same time by a camera system. As a specific example, the A fiducials 704 and the C fiducials 704 of FIG. 7H can be within the minimum spacing of each other because their cones of visibility are non-overlapping. In an example, this operation 762 can involve defining the problem as a constraint satisfaction problem and solved using any of a variety of known techniques for doing so (e.g., backtracking, constraint propagation, local search techniques, other techniques, variations thereof, and combinations thereof).

Operation 766 includes embedding the individual fiducials 704 in hollows 710 to reduce multi-face visibility at high angles. This can be a manufacturing step or a design step whereby the desired parameters of the hollows 710 is determined.

Operation 768 includes tuning the hollows 704 and navigation software (e.g., camera-to-face angle) so face switching occurs before mechanical occlusion of fiducials by the hollows occurs. For instance, a navigation system can be configured to detect an array of fiducials defined by individual fiducials having coordinates in 3D space and parallel normals. The array of fiducials can be associated with a maximum-angle parameter that describes how angled a camera normal can be relative to the normals of the fiducials while still being a valid match. This can be because at sufficiently large angles, the apparent distance between the fiducials of the array becomes small or skewed in a manner that reduces navigation accuracy to too great of a degree. The value of that parameter can be set to be less than the cone of visibility of the associated array of fiducials (e.g., as defined by the hollows of a pod). In other words, the parameter can be configured such that software defined face switching occurs within the overlap zone 792 of the cones of visibility 790. This can reduce the occurrence of a situation from occurring where no valid array of fiducials is visible.

The specific face switching behavior can be specified by the navigation system. For instance certain navigation systems can permit a single object (e.g., an end effector of a robot) to be tracked using multiple different arrays of fiducials. In some examples, tracking can occur simultaneously such that each array contributes the same or weighted amounts to the determined location of the tracked object. In other examples, only one array contributes to the determined location at a time.

Operation 770 includes measuring the fiducial center optically and create custom navigation files to reduce optical fitting errors by the localization system. This can be a calibration step used to improve the accuracy of the system.

Operation 772 includes manufacturing the designed surface 702.

There is a mechanism for anchoring and sensing drape attachment to the robotic end-effectors.

In some medical device applications where optical clarity of an instrument is beneficial (e.g., imaging, surgical tool navigation and robotics, etc.), it can be beneficial to stretch the sterile barrier (e.g., drape) over the surface of the device and resist wrinkling the drape during the procedure. This section describes a mechanism that provides a drape anchor anchoring (e.g., to hold a stretched drape in position) and sensors for detecting the presence of the drape anchors at various locations and provide a signal to the system and user if a drape is moved or not properly installed.

A drape and body system as described above where the drape has anchoring elements attached to predetermined locations on the inside surface of the drape. The locations can be selected to discourage unwanted bunching or wrinkling of the drape. The non-sterile body has pockets for anchor elements within retainers. There can be reflective optical sensors at the bottom of the pockets. The pockets can be placed at locations on the body with sufficient hoop stress on the drape when the drape is properly stretched over the body. Stress from drape material can urge the anchor elements against the surface of the body and when drape has reached its designed travel distance and the anchors drop into the pockets and are retained in place against moving back on the body. Optical sensors can sense infrared or other light reflecting back off the anchors bottom surface and only when they are fully dropped in they provide positive signal.

FIG. 8A illustrates a body 802 covered by a drape 804. In the illustrated example, the body 802 is a non-sterile robot end effector (e.g., of robot 500), but disclosed examples can be applied to other bodies to be draped. The drape 804 has anchors 806 and a retention ring 808. The body 802 includes a capture area 810 configured to retain the anchors 806. The anchors 806 can be retained by the capture area 810 such that the retention ring 808 can be pulled proximal to the anchors 806 such that the anchors 806 resist distal movement of the retention ring 808 (e.g., at least in part because the capture area 810 resists distal movement of the anchors 806). The anchors 806 can hold the retention ring 808 such that the drape 804 is stretched over the body 802. The stretching can be sufficient to usefully resist the drape 804 from bunching, wrinkling, or otherwise being disposed in such a way as to interfere with operation of the robotic end effector (e.g., bunching in a way that interferes with active or passive infrared fiducials). The body 802 (e.g., a robotic end effector) can include one or more sensors 820 configured to detect the presence or absence of the anchors 806 within the capture area 810.

The anchors 806 and retention ring 808 can be part of the drape 804 in any of a variety of ways such as by gluing, welding, attaching, making integral, other techniques, or combinations thereof.

The anchors 806 are components configured to facilitate retain the drape 804 relative to the body 802. In the illustrated example, the anchors 806 are plastic discs coupled with the drape 804. In other examples, the anchors 806 can be or have other geometries including but not limited to n-gons (wherein n is any whole number). The anchors 806 can be constructed from any suitable material. In this instance, the anchors 806 are configured to facilitate detection by the sensors 820. For instance, where the sensor 820 is an optical sensor, the anchors 806 can have material properties (e.g., reflectivity, coloring, opacity, other properties, or combinations thereof) selected to enhance detection of the anchors 806 by the sensor 820. In other examples, the sensor 820 is a force sensor and the anchor 806 can be configured (e.g., shaped) to exert a specific kind of force over a particular area (e.g., a force in part provided by the drape being stretched). In other examples, the sensor 820 is a Hall Effect sensor and the anchor 806 is configured with one or more magnetic components to facilitate detection by the sensor 820. In still further examples, the sensor 820 is an open circuit and the anchor 806 is configured to close the circuit in a detectable way.

In the illustrated example, the retention ring 808 is an annular component coupled to the drape 804. The retention ring 808 can be configured to stretch the drape 804 in a useful way. For example, in the illustrated example, the retention ring 808 has a greater surface area in contact with the drape 804 compared with the anchors 806. That surface area of the retention ring 808 in contact with the drape 804 can be circumferentially disposed around the drape 804 to facilitate useful manipulation and stretching of the drape 804 by the retention ring 808.

FIG. 8B illustrates the anchor 806 disposed within a capture area 810 of the body 802. FIG. 8C illustrates a partial cross-section view of the capture area 810 taken along line C-C of FIG. 8B. As illustrated, the capture area 810 holds the anchor 806 such that the capture area 810 resists distal movement of the anchor 806. The ring 808 is disposed proximal to the anchor 806, which resists distal movement of the ring 808.

The capture area 810 defines a lead-in groove 812 and a pocket 814. A sensor 820 is disposed such that the sensor 820 can detect the presence or absence of the anchor 806 within the pocket 814.

The capture area 810 defines a lead-in groove 812. The lead-in groove 812 is disposed distal to the pocket 814 of the capture area 810. The groove 812 is configured to guide the anchor 806 into the pocket 814. The pocket 814 can be a recessed area of the body 802 designed to receive the anchor 806.

The sensor 820 can be any of a variety of one or more sensors configured to detect the presence of the anchor 806 within the pocket 814. In many examples described herein, the sensor 820 is an optical sensor. The sensor 820 can be configured to sense infrared light. The source of the infrared light can be a component of the sensor 820, a component of the body 802, or another source. In other examples, the sensor 820 can be a Hall Effect sensor, a force sensor, a circuit, another sensor, or combinations thereof. There are benefits to using an optical sensor. Optical sensors can provide reliability benefits. Further, the use of optical sensors can reduce the cost of disposables (e.g., the drape 804 and anchor 806).

The sensor 820 can have a predetermined focal length 822. The front face of the sensor 820 that faces the pocket 814 is disposed at an angle 824 relative to the bottom of the pocket 814. The focal length 822 and angle 824 can be selected and predetermined to increase the ability of the sensor 820 to detect the anchor 806. In an experiment, the sensor 820 was configured as an infrared sensor and produced an output of 0.05-0.1 volts in ambient light. 0.1-0.2 volts when covered by the drape only, and 4.2 volts when being covered by the drape angle. A controller can be configured using such voltage outputs to provide useful output indicating whether a drape is present or not.

The output of the sensor 820 can be used by an intraoperative system to help a user determine whether the body 802 is correctly draped and provide an associated output. For instance, using the output of the sensors 820, a system can alert a user and describe which of the anchors is not properly placed.

Traditional surgical navigation systems can suffer from interference between adjacent markers on separate tracking faces of a multi-face tracking array. Such interference can constrain the mechanical design of arrays and other components. While some examples above address this issue using fiducials 704 sunk within hollows 710, in addition or instead, examples described in this section can be used.

Examples described herein are relevant to addressing such interference problems by via selective face switching. A selective face switching process can include a controller configured to cause a subset of all faces to be active at a given time (e.g., in response to a trigger signal) according to a schedule. Such a switching process can ensure that no two adjacent markers are active for any given frame, there would be no risk of marker interference between nearby fiducials 704 of different faces 510.

In some examples, the schedule for face activation is a simple schedule such that the faces take even turns in being active (e.g., face one, face two face three, and then back to face one and so on such that the fiducials 704 each face 510 are active for 1/n activations where n is the total number of scheduled faces). In other examples, certain faces 510 are active for more than an equal share of the time. For instance, faces 510 can be prioritized for activation based on surgical requirements. For instance, faces 510 that are physically closer, not obscured, or otherwise more relevant to a current phase of an operation (e.g., a current spinal level or pedicle trajectory) are prioritized (e.g., such that they account for more than 1/n activations). In addition or instead, an accelerometer can be used to prioritize faces 510 that are opposite the gravity vector with the assumption that the camera typically resides above the tracking face 510. Such prioritization can improve accuracy of the tracking system (e.g., by providing more rapid updates for fiducials covering more important faces).

FIG. 9A illustrates a surgical tool 902 having a controller 904, a first face 910, a second face 920, and a third face 920. The first face 910 has a first pattern of active fiducials 912. The second face 920 has a second pattern of active fiducials 922. The third face 930 has a third pattern of active fiducials 932. Each face 910, 920, 930 has an associated activation sensor 940.

The surgical tool 902 can be any surgical tool of interest to be tracked during a surgical procedure by a surgical navigation system (e.g., as described elsewhere herein). Examples of surgical tools 902 include an end effector or another portion of a surgical robot, an instrument, an implant, another tool, or combinations thereof.

The controller 904 is a set of one or more components that control the active fiducials 912, 922, 932, such as by being able to selectively active and deactivate the fiducials. In an example, the controller 904 is configured to receive data or signals from the activation sensor 940, process the data or signals, and activate one or more fiducials according to a schedule based on the received data or signals. The controller 904 can be a dedicated controller for controlling the fiducials. In other examples, the controller 904 is part of a larger system that performs other functions as well. In some examples, the controller 904 can be a microcontroller. In addition or instead, the controller 904 can be or be part of a computer having one or more processors, memory, and other components as described elsewhere herein.

In an example, all fiducials 704 of a face 510 having an activation sensor 940 that was activated are activated. Thus, faces 510 that are not oriented toward activation source (e.g., camera) are not activated.

The faces 910, 920, 930 are logically or physically distinct regions of the surgical tool 902 having fiducials thereon.

The fiducials 912, 922, 932 are one or more activatable and deactivatable components used by a surgical navigation system for tracking. The fiducials 912, 922, 932 can be active infrared lights, passive infrared reflectors selectively blockable by shutters or other active components, active radiofrequency components, other active emitters, other blockable passive reflectors, other components, or combinations thereof. The fiducials 912, 922, 932 can correspond to the fiducials 704 described elsewhere herein and vice versa.

The one or more activation sensor 940 are a set of one or more components that detect a signal indicating that fiducials should be activated. The activation sensor 940 can be electrically coupled to the controller 904 such that the controller 904 can determine from the activation sensor 940 that the signal has been received. The signals can come in any of a variety of forms and the sensors 940 can be configured to detect such signals. In some examples, the signals are in the form of an audio frequency that is within (e.g., approximately 20 Hz to 20 kHz), above (greater than approximately 20 kHz), or below (below approximately 20 Hz) the typical human hearing frequency range and the activation sensor 940 can be configured to detect such a signal. In some examples, the signals are in the form of signals on the electromagnetic spectrum (e.g., ultraviolet light, visible light, or infrared light) and the activation sensor 940 can be configured to detect such signals. In some examples, the signals can be data signals sent from another component of a navigation system (e.g., in the form of networking packets communicated using BLUETOOTH, WI-FI, Ethernet, other technologies or combinations thereof).

Some existing navigation systems are configured to send infrared signals in the form of chirps to trigger active markers. For example, the POLARIS VICRA by NDI sends 20 Hz chirp signals that cause all active markers to emit infrared light for an integration time.

FIG. 9B illustrates a chart of example signals over the course of three frames (n, n+1, and n+2). The signals are visualized as being either high or low. There are signals associated with the activation sensors 940, such that a high signal indicates that the particular activation sensor 940 detected an activation signal 906. The activation sensor signals are labeled A₁, A₂, and A₃, respectively corresponding to the first faces 910, the second face 920, and the third face 930. There are also fiducial activity signals associated with the groups of fiducials 912, 922, 932, such that a high signal indicates that the particular fiducials of that group are active and that a low signal indicates that the particular fiducials of that group are not active. The fiducial activity signals are labeled F₁, F₂, and F₃, respectively corresponding to the first group of fiducials 912, the second group of fiducials 922, and the third group of fiducials 932.

In some implementations, the chart of FIG. 9B illustrates signals going to and coming from the controller 904. For instance, the activation sensor signals can represent data coming to the controller 904, and the fiducial activity signals represent control signals being sent from the controller 904 to activate particular groups of fiducials. The controller 904 can selectively activate the fiducials based on an algorithm or schedule, such as is described above.

FIG. 9C illustrates the surgical tool 902 during and shortly after the first frame. As illustrated, all activation sensors 940 receive and detect the activation signal 906, the first group of fiducials 912 activate. None of the other groups of fiducials 922, 932 activate.

FIG. 9D illustrates the surgical tool 902 during and shortly after the second frame. As illustrated, all activation sensors 940 receive and detect the activation signal 906, the second group of fiducials 922 activate. None of the other groups of fiducials 912, 932 activate.

FIG. 9E illustrates the surgical tool 902 during and shortly after the third frame. As illustrated, all activation sensors 940 receive and detect the activation signal 906, the third group of fiducials 922 activate. None of the other groups of fiducials 922, 932 activate.

Robot motion can be commanded via an external interface (e.g., human-machine interface, end effector, other interfaces, or combinations thereof) that receives input from a user. Motion of a robot can begin and end without requiring further interaction from the user. If the user wants the robot to stop moving, engaging any part of the robot arm with minimal force can stop the motion.

Previous solutions for engaging robot motion was performed with active operation of a user-interaction signal (e.g., a foot pedal or button). Surgeons have one or more foot pedals for various instruments in the operating room. Having a foot pedal for the robot adds to the clutter on the operating room floor and the confusion on which pedal should be used at any given time. Removing this reliance allows for a more intuitive workflow.

Removing the reliance on a foot pedal declutters the floor and cognitive burden on the surgeon. Also, not requiring continuous activation of a signal allows the user to focus on other aspects of the procedure. Stopping robot motion by minimal force applied anywhere on the robot arm, gives greater flexibility to the user and increases safety from collisions with critical structures.

In an example, a portion of a robot arm includes one or more buttons or other features for receiving a command from a user. The robot arm also includes one or more safety features (e.g., torque limiters, force sensors, etc.) configured to facilitate the safe use of the robot arm. In an example method, the robot receives a command from a user. The robot executes the plan (e.g., by moving the robot arm to a location) without requiring further input from the user (e.g., without requiring a foot pedal or other user interface element to be actuated). While executing the plan, the robot detects that one or more safety features has been tripped or otherwise activated. In response to that detecting, the robot ceases execution of the command.

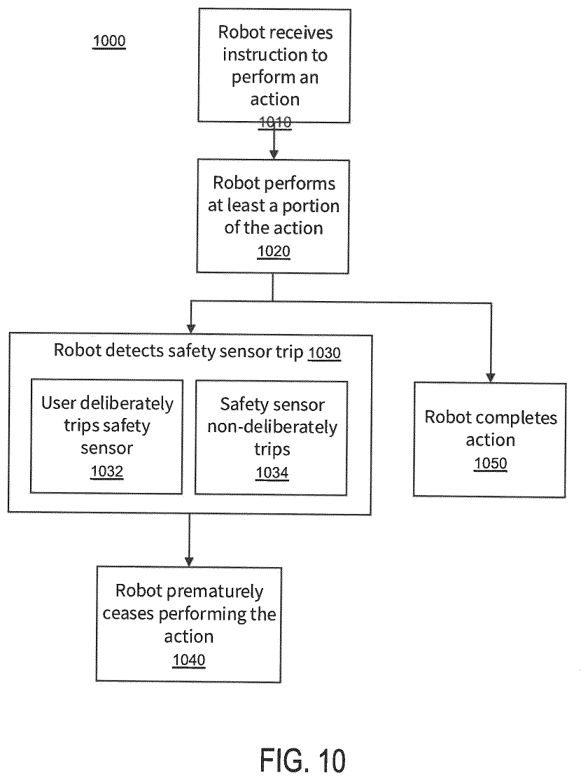

FIG. 10 illustrates an example method 1000. The method 1000 begins with operation 1010. In operation 1010, a robot receives a user command to perform an action. The action can be moving the robot arm to a particular location or to begin movement according to a predetermined plan (e.g., a surgical plan). In operation 1020, the robot performs at least a portion of the action. In an example, during operation 1020, the user provides no input (e.g., the user does not depress a foot pedal or actuate a hand or presence sensor of the robot arm) to the robot while the robot executes at least a portion of the action. In operation a20, the flow of the method can go to operation 1030.

In operation 1030, the robot detects a safety sensor trip. For example, detecting the trip can include determining that the value of a safety sensor is outside of a predetermined range, has been outside of a predetermined range for more than a predetermined amount of time, that the safety sensor indicates a trip state, that a specific signal has not been detected for more than a predetermined amount of time, that an accuracy is below an acceptable value, other determinations, or combinations thereof.

Operation 1030 can be for any of a variety of reasons. For example, 1030 can be the result of operation 1032 or 1034.

In operation 1032, a user deliberately caused a safety sensor of the robot to trip. For example, the user can apply force to a force sensitive region of the robot (e.g., pressing on the arm, thereby causing a force sensor of a joint of the arm to detect force that is above a predetermined safe threshold for the action), the user can deliberately block a sensor of the robot (e.g., an optical sensor), the user can deliberately cover a portion of the robot (e.g., block an active or passive infrared fiducial used to navigate the robot), the user can bump an object (e.g., the robot arm, a sensor thereof, a tracking system thereof, the patient, or other thing in the surgical area), the user can tug a drape, perform other actions, or combinations thereof. The user can deliberately induce activation of a safety sensor or feature of the robot despite otherwise safe operation of the robot. For instance, the user can trip a safety sensor of the robot even though the robot was acting safely and the user wanted the robot to stop for a non-safety related reason.

In operation 1034, a safety sensor of the robot trips in a non-deliberate way (e.g., a user of the robot did not deliberately induce the sensor to trip). For example, a safety sensor may detect that the robot is experiencing unwanted force (e.g., because the robot arm, drape, or other component is caught on or bumping into something), navigation is compromised (e.g., due to a blocked or bumped sensor), or something is otherwise occurring that violates a predetermined safe state or threshold.

Following operation 1030, the flow can move to operation 1040 in which the robot prematurely ceases the action.

In operation 1050, the robot completes the action.

Using a sterile interface panel (e.g., on the robot cart, end effector), the robot can receive user interactions directly without a separate non-sterile user input receiver.

In traditional implementations, a non-sterile surgical cart operator is required to allow a sterile user to manipulate surgical cart software. This interaction is time-consuming and can lead to user-annoyance and mistakes. If no non-sterile operator is available, a sterile user may break sterility to manipulate the cart software. This interaction adds unwanted time to the procedure. A sterile drape over the surgical cart is one solution, but the drape can occlude the view of the software and may interfere with touch-based control.

In an example, one or more user interface elements can be provided within or accessible within a sterile field for use in providing commands to a device disposed outside of the sterile field. Such commands can include but need not be limited to: commanding robot to next position, turning on hand guiding modes, selecting a next screw/step to perform, opening/closing camera view, minor plan adjustments for screw placement, capturing screw plan, commanding robot to move out of the way for C-arm imaging or patient access, turning on/off retractor lights, turning on/off powered tool, reorient robot pose, other actions, or combinations thereof.

In an example, one or more user interface elements are disposed on an arm of a surgical robot. The user interface elements can include one or more optical sensors, cameras, buttons, touch screens, touch sensitive strips, force sensitive strips, other user interface elements or combinations thereof.

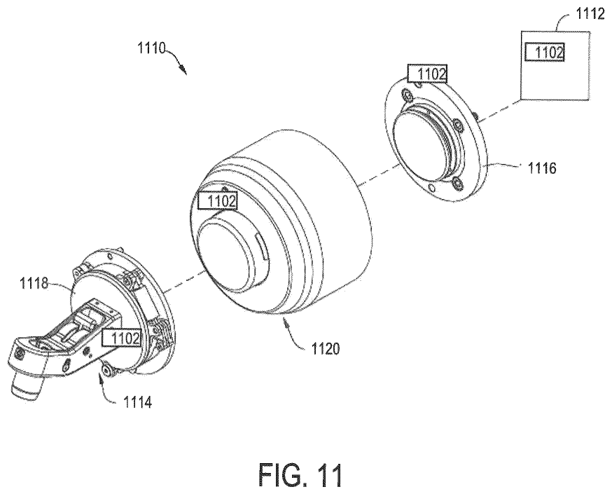

FIG. 11 depicts a connector assembly 1110, which generally can be used for connecting or otherwise securing a robotic arm 1112 (shown schematically in FIG. 11) and a medical end effector 1114 (e.g., a tool, a spinal tool, a tool holder, an instrument holder, combinations thereof, and/or the like). The connector assembly 1110 may include a plate 1116 that can be attached to the robotic arm 1112, for example using one or more fasteners. The connector assembly 1110 may also include an attachment assembly 1118 that may be integral with or otherwise secured to the end effector 1114. Securing the end effector 1114 to the robotic arm 1112 may be accomplished by securing the attachment assembly 1118 to the plate 1116. In some instances, a sterile barrier adapter 1120 can be disposed between the plate 1116 and the attachment assembly 1118. The sterile barrier adapter 1120 may help to facilitate separation of objects inside and outside of the sterile field. As illustrated, one or more user interface elements 1102 can be disposed on, in, or in relation to any of the components described above.

Manual adjustment of a planned trajectory can be beneficial in resisting skiving. Using hand guiding this adjustment can be made via the robot system using constraints. Often changes to a planned trajectory are required intraoperatively. Currently, these changes are done via a pointer or touch-based screw planning. Using a pointer requires the robot to be cleared from the surgical site, and using touch-based planning requires interaction with a user or the surgeon breaking sterility.

Rather than using an external mechanism for replanning, the user can hand guide the already positioned robot to make the adjustments required to continue with the procedure. Two example modes of adjustment are: angular pivoting or planar motion. Angular pivoting can provide the ability to create a pivot point relative to the tool center point to allow rotations about this point while maintaining the current position. This pivot can have virtual constraints to prevent certain angulations. Planar motion can provide the ability to create a plane normal relative to the tool center point to allow translations along the plane, but no rotations or translations off plane. This plane can have virtual constraints.

Using navigation the surgeon can see how these adjustments can affect the current trajectory. Once the adjustment is done, the robot is already in the correct place and the procedure can continue without any other interaction. The different control modes allow for minor adjustments to the robot position while maintaining certain elements of the translation/rotation of the control point. This blends together the precision of the robot arm and the control of a user.

Hand guiding is a method for manual manipulation of the robot arm by a user. This manipulation can occur at the joint level and/or in Cartesian spaces and can be constrained with limits and/or degrees of freedom. Creating and designing discrete arm poses and motions to satisfy all clinical conditions is challenging and unoptimized for a seamless surgical experience. Unpredictable or unanticipated robot motion can lead to distrust of the system.

There are various mechanisms that can enable manual manipulation of the robot arm. In an example, there is a state-based mechanism in which one or more interactive buttons are present on the robot arm or end effector. The one or more buttons can be triggered to enable hand guiding (e.g., manual manipulation) of the arm in a selected mode. Once this mode is active the user may manipulate the robot arm in the current hand guiding mode enabled. The robot arm can turn off hand guiding mode by detecting that there has been no system motion for more than a predetermined amount of time or by receiving user input that turns off the mode.

In another example, there is a continuous activation mode in which an interactive button present on the robot arm or end effector must be continuously held (or otherwise actuated) to enable hand guiding.

There can be various hand guiding modes including but not limited to: 6 DOF (Degrees of Freedom), 6 DOF constrained, lift column, linear translation, angular pivot, and planar.

In 6 DOF mode, the system provides a user with the ability to manipulate the robot arm via a combination of individual joints (impedance mode) and the end effector (admittance mode). This mode allows the tool center point to be moved with 6 degrees of freedom within the workspace of the robot. Motion through a singularity is prevented or smoothly transitioned through by limiting the overall velocity of the joints dynamically.

In 6 DOF constrained mode, the system provides similar capabilities to 6 DOF mode, but with an addition of virtual workspace constraints to protect critical structures. These virtual constraints can take the form of any shape/size and act as keep out zones for the tool center point and components of the robot arm. This is used to prevent contact with tracking arrays, surgical equipment, the patient, and other critical structures. The constrained motion can also be along a defined path.

In lift column mode, the system provides a user with the ability to translate up/down force on the end effector to motions to raise/lower the robot base.

In linear translation mode, the system provides the user with the ability to create a trajectory along an axis relative to the tool center point and use measured force along that direction to move the tool center point along a linear line with or without virtual end points.

In angular pivot mode, the system provides the ability to create a pivot point relative to the tool center point to allow rotations about this point while maintaining the current position. This pivot can have virtual constraints to prevent certain angulations.

In planar mode, the system provides the ability to create a plane normal relative to the tool center point to allow translations along the plane, but no rotations or translations off plane. This plane can have virtual constraints.

Hand Guiding can be used to allow the surgeon to manipulate the robot arm to a pose to perform a procedure (e.g., retractor management, pedicle screw placement, interbody placement). The different control modes allow for minor adjustments to the robot position while maintaining certain elements of the translation/rotation of the control point. This blends together the precision of the robot arm and the control of a user.

A system can provide a user with the ability to clear a robot from surgical site with single gesture to allow for quick access to the patient. The enables other surgical equipment/instruments to be used without interference from the robot. It also allows for manual intervention of the procedure. Using a single gesture (e.g., button press, gesture, voice command, etc.) a user can command the robot system to clear the surgical site. This moves the robot arm (not robot cart) to a position that is cleared from the surgical site but can be quickly deployed to continue the procedure. This move may or may not release the immobilizers that allow the robot cart to be moved in cases that require it. Beneficially, this technique adds to surgical efficiency, allows for other capital equipment (e.g., a C-arm imaging system) to be used adjacent to the robot system, and allows for quick access to the patient in emergency situations without a user physically moving the robot cart.

During a navigated surgical procedure, the patient and target surgical site can move and shift. When this happens, the user is alerted of the amount of motion and instructed to use a gesture to readjust the robot. The readjust motion can depend on the amount of patient motion. Patient motion is inevitable during a surgical procedure. Assuming no motion and not correcting for motion could lead to inaccuracies. Alerting a user of motion without providing a way to correct for the motion may cause user annoyance. The correction motion and feature should be fast, safe, and efficient otherwise delay in surgery and user annoyance can occur.

During a navigated surgical procedure when the robot system is in position in the target surgical area, the stable location of the patient is cached and monitored against. On the UI screen or via another user interface panel (e.g., a light ring on an arm of the robot) the relative displacement of the surgical site during the procedure is shown to the user. When this displacement exceeds a certain limit the system instructs the user to readjust the robot. When the robot readjust gesture is activated, if the displacement is less than a predetermined threshold distance, then the robot adjusts in place. Otherwise, the robot retracts along its trajectory to a safe distance then readjust and move back towards the surgical site

The ability to readjust the robot allows for higher accuracy by compensating for inevitable motion due to the procedure. For small displacements, the robot adjusts in place to ensure surgical efficiency. For large displacements, the robot retracts to a safe area before readjusting to ensure no contact with critical structures (useful for large patient motions, changes in bed height, etc.).

When the robot system detects a collision with an object, the robot arm can stop and induce compliance so to avoid damage to the object or the robot system. This induced compliance also ensures the robot does not remain in a collision event. When the robot system is in motion and collides with an external object (patient, screw towers, equipment, staff, etc.) the robot can stop motion. If the external object is compliant or can be removed, then the system can begin motion again. However, if the external object is not compliant or cannot be cleared the robot remains in collision and could damage the external object. In cases where the external object is a patient or a critical structure attached to the patient, this could cause unintentional harm.

The robot system is configured to detect a collision via its linkages or at the tool center point. For collisions detected at the linkages or joints the arm moves affected joints backwards in the opposite direction of the collision with a distance scaled with the amount of torque detected, and apply compliance to those joints to allow for small reconfigurations of the arm to clear the collision(s).

For collisions detected at the tool center point, the arm moves in the opposite direction of the resultant force a distance scaled with the amount of force/torque detected, and apply compliance along the axis of the resultant force to allow for small reconfigurations of the arm to clear the collision(s).

Using a single-gesture the user is able to advance the procedure to any pedicle. This gives flexibility to the user to perform the procedure or subsets of the procedure in any order. Currently, users select a predefined order of pedicles to execute the pedicle screw implantation. This instructs the robot to move to the pedicle screws in this order. In order to go to another pedicle, the user navigates to all pedicles between the current pedicle and the target which can cause a delay in the procedure. Using a single gesture (clicking on next pedicle on a navigation system or a button on the end effector) the user can select the desired next pedicle to perform. When commanded to move the robot arm moves to the selected pedicle and ignore the set order. Upon completion of that pedicle, the next robot motion follows the set order from where it last left off or the user can select the next pedicle to perform.

User has the ability to prescribe their own workflow. Pedicle screws can be skipped if they do not need to be performed after intra-op evaluation. This can increase operating room efficiency when two users are present.

Due to the variability of operating rooms and the equipment used, having a robot system with the capability of being placed in multiple clinically relevant positions is useful in not disrupting efficiency. An example robot system can perform the prescribed procedure independent from which position is used. Prescribing a strict robot cart placement forces the operating room to be setup in a specific way. Many operating rooms cannot accommodate a single robot position due to other capital equipment, size, and procedures.

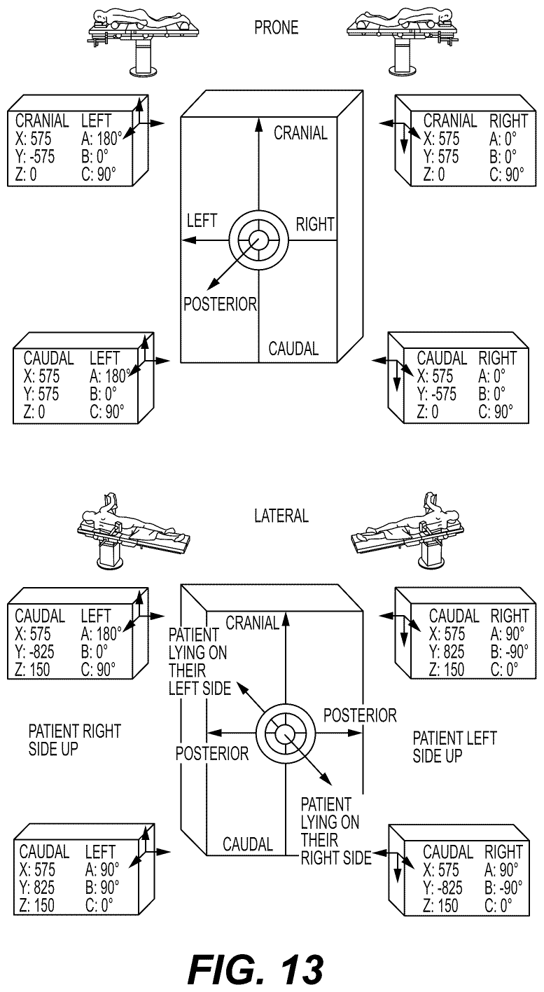

FIG. 13. illustrates example placements of a robot relative to a patient in the prone and lateral positions.

The system can be configured for such placement in any of a variety of ways. For example, the system can include multiple robot arms (e.g., in mirrored arm configurations). The configuration can be achieved by performing arm reach/collision detection for planned motions using all desired configurations for flexible operating room set up. The configuration can be achieved by the robot cart supporting kinematics of the robot arm to achieve similar results independent of robot position. The configuration can be achieved by mounting the robot arm on a side agnostic angle (e.g., 12 degrees) on the robot cart to ensure arm reach and ensure insignificant bias to one side or another

A user interface element with backend algorithm(s) prove real-time information related to the depth of an tool or implant compared to the plan. This allows the user to quickly comprehend the depth left to execute for a particular step. For example, how far left to drill a pilot hole, make a cut, or place a screw. User interface embodiments can vary, but can convey the proximity of the actual implant to the planned location of that implant.

There is a desire for improvements on current methods to communicate positional reality of a tool or implant to the final planned location of that tool or implant. Knowing where tools/implants are relative to anatomy and plan is beneficial for safe execution of pedicle screw fixation. Depth is one aspect of this information and can help mitigate breaches or ensure adherence to a plan for final screw placement.

Example user facing techniques for conveying depth include: a numerical readout indicating depth of the tip of an instrument relative to a goal location, filling an arc with colored indicators of “good”, “ok”, or “bad” depths, logic to show/hide elements based on a variety of factors (ex. tool visibility, depth distance, angulation), handling of negative depth, presenting a “bullseye view” that abstracts other live/plan tool axes focusing the user on depth.

Examples of backend techniques include: calculation of decomposed positional differences of two bodies in a single space. Whereas a single delta decomposed axes corresponds to the “live” tool's tip along the primary axis and the planned tool's tip along its primary axis. Another example is conditional and corrective logic to handle limit situations (ex. angular or distance). Another example is smoothing and or rounding methods to ensure a positive user experience.

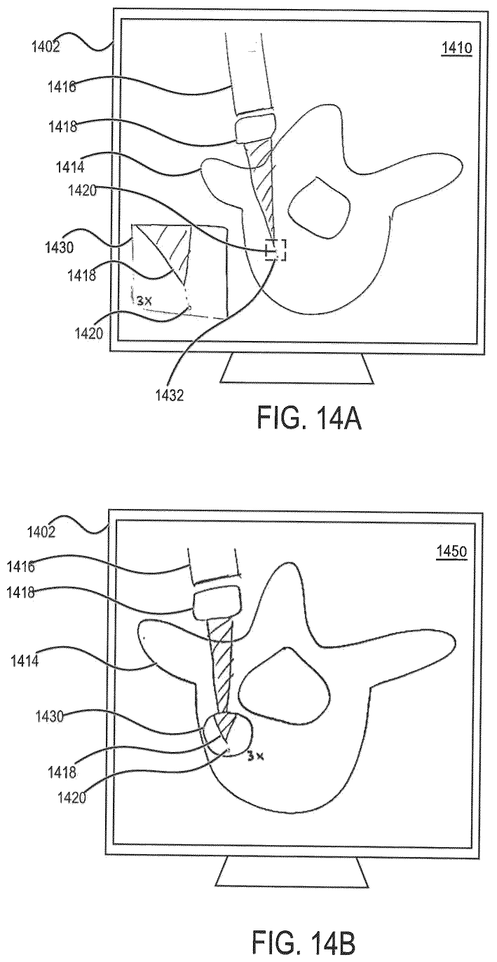

FIG. 14A illustrates a display 1402 providing a first user interface 1410 showing a representation of a vertebra 1414 having a representation of an instrument 1416 and an implant 1418 disposed relative thereto. The first user interface 1410 further illustrates a target location 1420 for the implant 1418 to reach to satisfy a predetermined plan. To facilitate the user's understanding of how close the tip of the implant 1418 is to the target location 1420, the first user interface 1410 provides a magnified representation 1430. As illustrated, the magnified representation 1430 displays an enlarged version of a region of interest 1432. Here, the region of interest 1432 displayed away from the region of interest 1432 in a way that does not obscure areas of importance. In the illustrated example, the magnification of the region of interest 1432 displayed within the magnified representation 1430 is at three times magnification. The magnification amount can be predetermined and can be customized by the user. In some examples, the magnification amount is scaled with the determined accuracy of a navigation system used to determining the position of the implant 1418 to avoid providing to the user a false sense of the accuracy of the true position of the implant 1418. The scaling can be based on a lookup table or based on an algorithm that takes into account a predicted distance that a user is from the screen and the apparent accuracy that the user would perceive given the true accuracy value determined by the navigation system. In some examples, the display of the magnified representation 1430 can be toggled via a user interface element. In addition or instead, the magnified representation is automatically displayed or hidden in response to a portion of the implant 1418 being determined to be in within a predetermined distance to the target location 1420.

FIG. 14B illustrates the display 1402 providing a second user interface 1450 showing a representation of the vertebra 1414. The second user interface 1450 is the same as the first user interface 1410 except the magnified representation 1430 is superimposed directly over the region being magnified.

In these examples, the target location 1420 is the location of a tip of the implant 1418 (e.g., a pedicle screw). But other target locations 1420 can be used and the region of interest 1432 can be shifted accordingly. For example, the target location 1420 could instead be the bottom of a pedicle screw tulip or head relative to planned locations for such components.

Other depth indicating techniques can be used, such as changing a color of lighting on an arm of the robot or elsewhere or audio ques (e.g., with frequency, tone, sound, or other audio characteristics changing based on depth).

Benefits of disclosed depth gauges can include: reduced mental load, consuming less user interface real estate, and reduce the likelihood of off plan executions.

The screw length confirmation algorithm allows the user to confirm that they are navigating and placing a pedicle screw with the planned screw length. It also evaluates proper screw attachment to a navigated screwdriver. This helps mitigate the risk of physically placing a different screw than what was planned and/or displayed on the navigation screen, and ensures a mis-attached screw does not get placed incorrectly.

The traditional workflow during pedicle screw placement requires manually updating the navigation software with the planned screw size and user confirmation that the planned screw is attached to the screwdriver. There is no software-based risk mitigation to prevent physically placing a different screw than what was planned and/or displayed on the navigation screen, potentially causing improper screw placement and complications for the patient.

In an example embodiment, before pedicle screw insertion, the user is prompted to place the assembled screwdriver on a navigated reference point (e.g., divot on a robot tool guide or confirmation block). The system then evaluates whether the screw attached to the screwdriver matches the planned screw length in the navigation software. If there is not a match, then the system determines whether the incorrect screw size is attached (e.g., the screw length is too long or too short) or if the screw is not properly assembled on the screwdriver. The appropriate result is then displayed to the user.

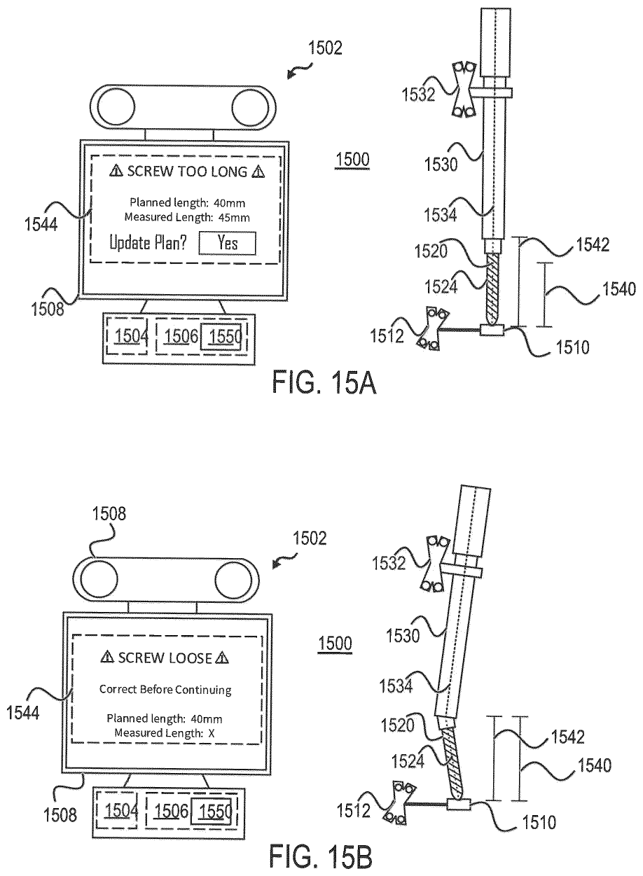

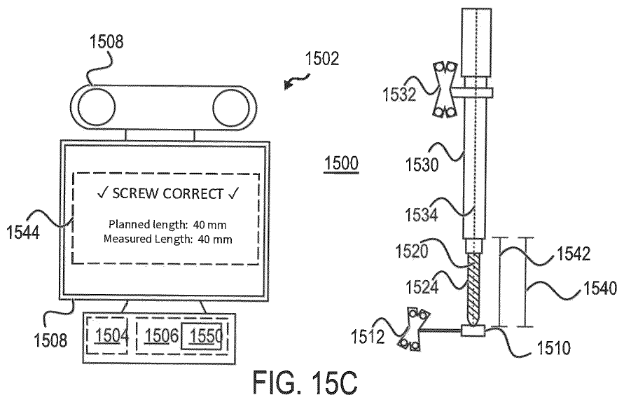

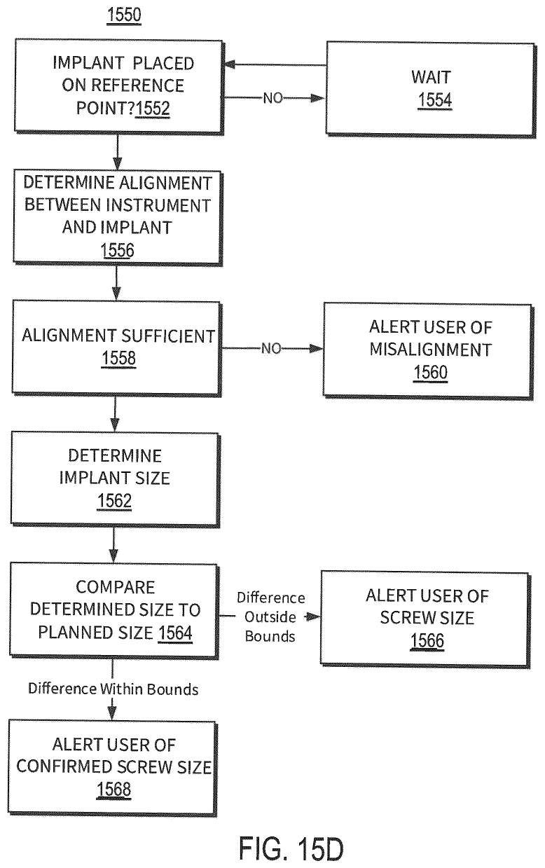

FIG. 15A-15C illustrate a surgical system 1500 including a surgical navigation system 1502 having an implant confirmation algorithm 1550 for confirming the appropriateness of an implant 1520 coupled to an instrument 1530 and placed in a reference 1510. The surgical navigation system 1502 can have one or more of the features and components as described in relation to system 100, above. As illustrated, the surgical system 1500 includes a processor 1504 and memory 1506 having stored thereon the implant confirmation algorithm 1550 (an example of which is described in more detail in relation to FIG. 15C).

As illustrated, the surgical navigation system 1502 includes one or more processors 1504, memory 1506 having instructions for the algorithm 1550 stored thereon, a display 1508. The one or more processors 1504 can be

The one or more processors 1504 can be one or more physical or virtual components configured to obtain and execute instructions. In many examples, the one or more processors 1504 are central processing units, but can take other forms such as microcontrollers, microprocessors, field programmable gate arrays, graphics processing units, tensor processing units, other processors, or combinations thereof.

The memory 1506 is one or more physical or virtual components configured to store information, such as data or instructions. In some examples, the memory 1506 includes a computing environment's main memory (e.g., random access memory) or long-term storage memory (e.g., a solid state drive). The memory can be transitory or non-transitory computer-readable or processor-readable storage media. The memory 1506 can include read only or read-write memory.

The instructions for the algorithm 1550 are one or more instructions that, when executed, cause the one or more processors 1504 to perform one or more operations for implant confirmation as described herein (an example of which is described in more detail in relation to FIG. 15C).

The reference 1510 is a component having a datum reference in which a portion of the implant 1520 can be disposed for useful implant confirmation by the system 1500. As illustrated, the reference 1510 can have a tracking array 1512 (e.g., including active or passive radiofrequency infrared fiducials) disposed in a determinable relationship with the datum reference so the system 1500 can determining the location of the datum reference.

The implant 1520 is a component configured for implantation in a patient's body and defining an implant axis 1524 (e.g., a long axis of the implant along which the implant 1520 is to be inserted into the patient). As illustrated, the implant 1520 is a spinal implant (specifically a cannulated or non-cannulated pedicle screw), but aspects described herein can be relevant to other medical implants. Other example components include intramedullary devices, cranial implants, spinal fusion implants, nails, blades, intervertebral implants, total disc replacement implants, plates, screws, spinal rods, structural rods, non-structural rods, spinous process implants, interlaminary spacers, standalone implants, rib-hook implants, piggyback implants, other implants, or combinations thereof.

The instrument 1530 is a component configured to facilitate implantation of the implant 1520 in the patient's body and defining an instrument axis 1534 (e.g., a long axis of the instrument along which the instrument is to be used to insert the implant into the patient). As illustrated, the instrument 1530 is a powered or manual driver for driving a pedicle screw implant to the patient's body. In other examples, the instrument can take any of a variety of forms and be configured to insert any of the implants 1520 into a patient's body.

The instrument 1530 is trackable by the surgical navigation system 1502. The instrument 1530 can include any of a variety of components to facilitate tracking. For instance, the instrument 1530 includes a tracking array 1532 (e.g., including active or passive infrared or radiofrequency fiducials) disposed in a determinable relationship with the instrument 1530. The instrument can have a length and/or geometry that it determinable by the surgical navigation system 1502. Thus, by tracking the position of the tracking array 1532, the surgical navigation system 1502 can determine the position of the region by which the instrument 1530 attaches to the implant 1520. Then by determining the position of the tracking array 1532 and the tracking array 1512, when the implant 1520 is seated in the datum of the reference 1510, the size of the implant (e.g., its length in a direction along the instrument axis 1534) can be determined. As a result, the system 1502 can warn the user if the actual size 1542 and expected size 1540 of the implant differ. Further, by determining the angle of the tracking array 1532 while the implant is seated in the datum, the surgical navigation system 1502 can determine the relative angle between the implant axis 1524 and the instrument axis 1534. Such an angle can be used to determine whether the implant 1520 is properly coupled with the instrument 1530. Thus the system 1502 can warn the user if an improper coupling is determined.

Referring specifically to FIG. 15A, the implant 1520 coupled to the instrument 1530 is shorter than planned. This difference in lengths and correctness of coupling can be determined by the surgical navigation system 1502 by executing the instructions for the algorithm 1550 on the one or more processors 1504. As a result of the difference between expected size 1540 and actual size 1542, the one or more processors 1504 cause the display 1508 to provide a notification 1544 alerting the user that the implant 1520 (in this illustration, a pedicle screw) is longer than planned. The notification 1544 further asks the user whether to update the plan. If the user responds that the plan is to be update, the planned length of the implant 1520 can be overridden with the measured size 1542. Then the surgical navigation system 1502 can continue with surgical navigation. In some examples, the surgical navigation system 1502 prevents a navigation process from continuing until the measured length corresponds to the plan. For instance, the system 1502 may prevent the showing of an unobstructed navigation screen or prevent advancement of navigation until the discrepancy is corrected (e.g., by updating the plan or by attaching and confirming the length of a new screw). In some examples, the notification 1544 can provide one or more suggested remediation actions, such as by attaching a specific screw or updating the plan.

Referring specifically to FIG. 15B, the implant 1520 is not properly coupled to the instrument 1530. This problem is detected by the surgical navigation system 1502 by executing the instructions for the algorithm 1550 on the one or more processors 1504. As a result, the one or more processors 1504 cause the display 1508 to provide a notification 1544 alerting the user that the implant 1520 (in this illustration, a pedicle screw) is not correctly coupled to the instrument 1530. The notification 1544 further instructs the user to correct the problem before continuing. In some examples, the surgical navigation system 1502 prevents a navigation process from continuing until issue has been corrected. For instance, the system 1502 may prevent the showing of an unobstructed navigation screen or prevent advancement of navigation until the instrument 1530 is properly connected to the implant 1520. In some examples, the notification 1544 can provide one or more suggested remediation actions based on a determined issue. For instance, the algorithm 1550 may determine that there is too much play between the instrument 1530 and the implant 1520 and instruct the user to tighten them. In some examples, the algorithm 1550 may attempted to also determine the length of the implant 1520 and provide an indication of the correctness of its size to the user. In other examples, the algorithm 1550 can wait until the connection between the implant 1520 and the instrument 1530 is correct.

Referring specifically to FIG. 15C, the implant 1520 is properly coupled to the instrument 1530 and the screw expected screw size 1542 and the actual screw size 1540 are the same. This is detected by the surgical navigation system 1502 by executing the instructions for the algorithm 1550 on the one or more processors 1504. As a result, the one or more processors 1504 cause the display 1508 to provide a notification 1544 alerting the user that the implant 1520 (in this illustration, a pedicle screw) is correctly coupled to the instrument 1530 and is the correct length. The notification 1544 may require the user to acknowledge the correctness (e.g., by actuating a user interface element) or may simply allow a navigation process to proceed.

FIG. 15D illustrates an example algorithm 1550 for implant confirmation. The algorithm 1550 can start with operation 1552.

Operation 1552 includes determining whether the implant 1520 is placed on the reference 1510. In an example, the reference 1510 includes a button, sensor, or other component configured to determine whether an object is disposed in a reference datum of the reference 1510. That determination can be sent to the system 1502 and used to determine that the implant 1520 is placed on the reference 1510.

The operation 1552 can include determining that both the instrument 1530 and the reference 1510 are trackable by the navigation system 1502. In some examples, the determination that the implant 1520 is placed on the reference 1510 can be determined based on tracking the movement of the instrument 1530. For example, the instrument 1530 can be tracked and observed to be moving in a way characteristic of the instrument 1530 being coupled to an implant 1520 (e.g., a distance from the instrument 1530 to the datum of the reference 1510 is greater than would be expected if an implant 1520 were not connected to the instrument 1530). Further, the instrument 1530 can be tracked and observed to be moving in a way characteristic of the implant 1520 being disposed within the datum. For instance, the instrument 1530 can be observed articulating about a point corresponding to the approximate location of the datum. Such determinations can be sufficient to determine that the implant 1520 is placed on the reference 1510.

In yet another example, the operation 1552 can be performed by asking for and receiving confirmation from a user that the implant 1520 is placed on the reference 1510.

If it is determined that the implant 1520 is not placed on the reference 1510, then the flow of the method can move to operation 1554.

Operation 1554 can include waiting for the implant 1520 to be so placed. In some examples, the system 1502 can instruct (e.g., via a message on the display 1508) the user to couple the implant 1520 to the instrument 1530 and to place the instrument on the reference 1510.

If it is determined that the implant 1520 is placed on the reference 1510, then the flow of the method can move to operation 1556.

Operation 1556 includes determining alignment between the instrument 1530 and the implant 1520. In an example, this operation 1556 includes determining the instrument axis 1534. The instrument axis 1534 can be determined in any of a variety of ways. In an example, the instrument axis 1534 is in a fixed relationship with the instrument tracking array 1532. The navigation system 1502 determines the location of the tracking array 1532, which is then used to infer the instrument axis 1534. The instrument axis 1534 can be represented in any of a variety of ways. In one example, the instrument axis 1534 is represented by two points in a three-dimensional coordinate space used by the navigation system 1502.

Once the instrument axis 1534 is determined, the implant axis 1524 can be determined. In an example, the implant axis is determined by drawing a line or defining an axis between the known location of the distal tip of the instrument 1530 (or the instrument axis 1534) and the known location of the datum of the reference 1510. The location of the reference point or a portion thereof can be determined using the tracking array 1512. In other examples, the implant axis 1524 is directly measured or observed using the navigation system 1502.

Once the instrument axis 1534 and the implant axis 1524 are determined, they can be compared to determine the alignment between the instrument and the implant. In some examples, angles between the axes 1524, 1534 are determined and used to determine the alignment.

In operation 1558 it is determined whether the alignment determined in operation 1556 is sufficient. There can be a predetermined acceptable alignment value between the axes 1524, 1534 and an acceptable amount of deviation (e.g., to account for manufacturing tolerances or navigation inaccuracy). In the illustrated example, the acceptable alignment is that the axes 1524, 1534 are coaxial (e.g., the axes 1524, 1534 define the same line). In other examples, it may be desirable for the axes 1524, 1534 to be angularly offset from each other (e.g., for antero-lateral approaches as described in relation to FIGS. 18-22 of U.S. Pat. No. 8,740,983, which was filed Nov. 12, 2010, and which is incorporated herein by reference in its entirety for any and all purposes). In some examples, the system 1502 can determine which implant 1520 and instrument 1530 are used and their acceptable alignment values.

If the alignment determined in operation 1556 is not sufficient, then the flow of the algorithm can move to operation 1560. In operation 1560 the user is alerted of the misalignment, such as is described in FIG. 15B. If the alignment determined in operation 1558 is sufficient, then the flow of the algorithm can move to operation 1562.

In operation 1562 the actual size 1542 (e.g., length or another dimension of interest) of the implant 1520 is determined. In an example, the actual size 1542 of the implant 1520 is determined by measuring a distance between a distal tip of the instrument 1530 (e.g., determined by the navigation system 1502 using the tracking array 1532) and the datum of the reference 1510 (e.g., determined by the navigation system 1502 using the tracking array 1512). In other examples, the actual size 1542 of the implant 1520 is measured, such as by using an optical camera.

Following operation 1560, the flow of the algorithm 1550 can move to operation 1564.

Operation 1564 includes comparing the actual size 1542 with a planned size 1540. In some examples, the planned size 1540 can be loaded from memory. If the difference between the actual size 1542 and the planned size 1540 is outside of bounds, then the flow of the algorithm 1550 can move to operation 1566. In operation 66, the system 1502 can alert the user of the difference in screw size, such as is shown in FIG. 15A. If the difference is within bounds, then the flow of the algorithm 1550 can move to operation 1568 in which the system 1502 alerts the user that the screw size is correct, such as is shown in FIG. 15C.

Traditionally, the parking location of the robot is not provided to the user, the user must place the cart along the bedside in the location that they think allows them to reach all pedicle screws in the plan. Once the user has placed the cart at the bedside, the user physically performs a dry run of the pedicles to ensure that all screws can be achieved with the chosen parking location. If the parking location is not sufficient the user repeats the steps listed above, which can be frustrating and time consuming.

In an improved example, a bedside docking algorithm provides an suggested parking location for the base of the robot cart, such that the cart operator can park the robotic cart once for a procedure. The calculated parking location is then displayed on the UI as a target area, once tracking is established with the navigation system, the robot cart's position is displayed in relationship to the target area. When the user starts to move the robot cart, the position is actively displayed on the user interface of the navigation system and guides the user to target area. With this algorithm, the system can provide a user with information for quickly and effectively parking the robotic cart at the patient bedside in a useful position for the procedure.

The bedside docking algorithm takes two inputs, processes them, and provides a set of output coordinates of the target parking location. The first input is a valid connection to a camera device that is capable of tracking an array on the robot cart and an array or arrays attached to the patient reference hardware. The second is a target area, which can include the location of the patient reference hardware in DICOM space and set the patient reference hardware coordinates with an applied offset, as the target area for the end effector once the base of the cart is docked at the patient bedside. If there is more than one patient reference hardware present due to the length of the operating region, then the algorithm can calculate the minimum number of parking locations to achieve every planned pedicle screw in the screw plan(s). During the process of bedside docking the user interface provides a visual representation of the target area, patient reference hardware, and actual location of the robot cart. The user interface updates dynamically as the user moves the cart, once the cart is in the target area bounds, the user interface indicates that the cart is in the correct location for executing all screws in the plan and inform the user they can proceed to clinical use.

With the bedside docking algorithm and the corresponding user interface can provide one or more of the following: the calculated bedside docking location, minimized cart movement during clinical use, visual representation of the target parking location, guidance to the target parking location, and confidence in chosen parking location.

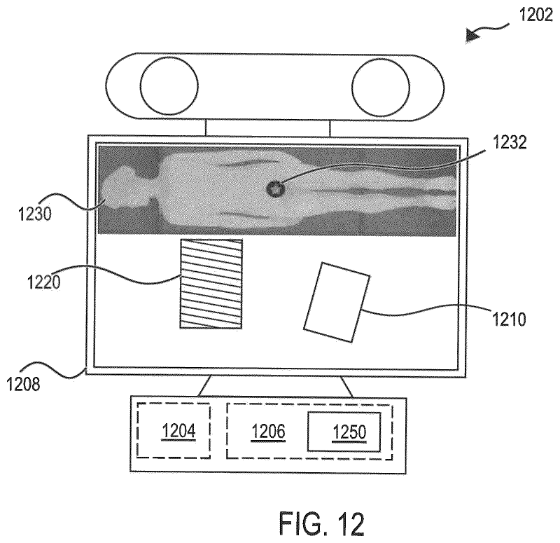

FIG. 12 illustrates a surgical navigation system 1202 configured to facilitate parking a robotic cart relative to a patient. The surgical navigation system 1202 includes a display 1208 showing a user interface that includes a representation of a robot cart 1210, a representation of a determined parking location 1220, a representation of a patient 1230, and a representation of a patient reference array 1232. As illustrated, the surgical navigation system 1202 includes a processor 1204 and memory 1206 having stored thereon a parking algorithm 1250 for causing the one or more processors 1204 to perform one or more parking operations.

The system 1202 can determine the location of the robot cart 1210 and the patient reference array 1232 (and thus the location of the patient) using navigation capabilities of the system 1202. The algorithm 1250 can determine the parking location 1220 using one or more processes as described above. The system 1202 can update the location of the components in real time to facilitate the user moving the robot to the parking location 1220.

Current robotic solutions in the field require manual interaction to immobilize the system. If this step is not performed, the integrity of the robot position can be compromised. Further, manual deployment/retraction of immobilizers can be forgotten leading to an impact on the integrity of the system. Also, manual deployment/retraction of the immobilizers are typically designed in a way that both a sterile/non-sterile user can use the sub-system.

Examples disclosed herein can automatically engage immobilizers or stabilizers of a robot cart, which can save time and improve the integrity of the robot position. A robotic system can automatically deploy immobilizers or stabilizers at specific steps in the surgical process, such as during screw case execution or robot registration steps. The immobilizers can be automatically retracted in all other parts of the workflow to allow for immediate egress of the system. An immobilizer retraction override can be used during screw case execution/robot registration if egress is required. By automatically deploying/retracting the immobilizers, an unintuitive procedural step is removed and a more simple user facing design can be implemented for emergency situations.

Example immobilizers or stabilizers can include those described in U.S. patent application Ser. No. 17/461,342, which was filed Aug. 30, 2021, and which was previously incorporated herein by reference.

During a navigated surgical procedure the patient and target surgical site can move and shift. When this happens the user is alerted of the amount of motion and if excessive enough instructed on next steps. Patient motion is inevitable during a surgical procedure. Assuming no motion, not correcting for motion, or not alerting a user could lead to gross inaccuracies. During a navigated surgical procedure when the robot system is in position in the target surgical area, the stable location of the patient is cached and monitored against. On the user interface screen or via another UI panel (e.g., a light ring on a robot arm) the relative displacement of the surgical site during the procedure is shown to the user. When this displacement exceeds a certain limit the user is alerted of excessive motion.

Limited arm compliance during pedicle screw placement is useful to maintain accuracy while instrumentation is being used. To increase the stiffness of the robot arm, the brakes for each joint can be controlled to maximize the overall stiffness.

Active position control with high stiffness often relies on delicate tuning of the control loop. External factors such as user interactions, weight of instruments, etc. can lead to arm motion that can result in inaccuracies. Even minor compliance in the control system can result in undesirable accuracy impacts.

Using a workflow-based approach, during positions when accuracy is not critical the brakes remain disengaged, however once accuracy is a primary goal of the position the robot is in, the brakes are engaged to remove any compliance from the control loop of the robot arm.

There are a variety of surgeon heights, table heights, and patient anatomy that is involved in spinal surgery. Due to the lack of consistency in these measures, creating a system that supports all configurations can be a challenge. If the height of the robot base is not optimized in the surgical working volume, then robot arm performance/execution may fail during use. Standardizing the robot base height with the patient bed height is one solution, however this can annoy the user and can create a less-than optimized ergonomic environment for the user.

The robot arm can be placed on a linear actuator allowing for vertical adjustment of the robot base between the operating room floor and ceiling. Based on the measured surgical environment conditions via navigation and other modalities, the optimized robot base height is computed and automatically applied to the linear actuator. As the surgical environment changes, a recomputed optimized robot base height is found and if a significant difference between the current and ideal height is found, then the height is automatically adjusted before the procedure continues to the next step.

Current solutions require manual control of the robot height or have no means to adjust robot height. By auto adjusting the robot base height the surgical working area for the robot and the ergonomics for the user are optimized.

Navigated surgery can include implant planning. This is often performed in spinal surgeries where pedicle screws are to be placed. Implant planning can include placing a representation of screw (e.g., a simple geometric shape meant to represent the screw or a complex geometric model of the screw) relative to one or more images of the patient's anatomy (e.g., two or three dimensional x-ray images or magnetic resonance imaging images). The user (often the surgeon) places the virtual screw in a desired position relative to the images of the anatomy. These images are then used as guides to facilitate placement of the actual implant close to the planned position. There are many different ways to specify the position of the virtual implant. Examples include the use of touch screens, keyboards, mice, or other input devices. However, intraoperatively it can be difficult to manage a sterile field while using such input devices. So one approach to screw planning involves a surgeon holding a navigated instrument relative to the patient's anatomy and the movement of the instrument in six degrees of freedom (e.g., pitch, roll, yaw, up/down, left/right, and forward/backward) causes a concomitant movement of the virtual implant in the plan. But it can be difficult to accurately move and hold the position of the implant.

An example implementation herein provides for improved gesture based planning using a pointer in the intraoperative field by artificially restricting the effects of movement of the instrument on the movement of the virtual implant. Using a navigated tool (e.g., a digitizer), a user can plan a screw trajectory pre-incision on a patient or post-incision in a patient. This navigated tool can be displayed on a visual representation of the patient.

For instance, the system can split the manipulation of the virtual implant into multiple stages. Each stage can be restricted in the kind of movement or manipulation of the implant is allowed. For instance, the system can monitor movement of the instrument in six degrees of freedom but only allow such movement to cause movement of the virtual implant in fewer than six degrees of freedom.

As a specific example, the system can provide for planning can in four stages: a trajectory stage, a depth stage, a width stage, and a length stage. The system can transition from one stage to another using any of a variety of different kinds of input. For instance, the instrument can be used to cause the system to move from one stage to another (e.g., by placing the instrument in a particular position or by actuating a button on the instrument), or voice commands can be used, or another user can directly interact with the navigation system to cause movement to another stage. Other inputs or combinations thereof can be used.

In a trajectory stage, the system uses gestures to change trajectory of an implant. In an example, during this stage, the navigation system monitors movement of the instrument in six degrees of freedom and causes movement of the virtual implant in six degrees of freedom. In some examples, the system monitors movement to cause movement of a virtual line in space and the line can be the basis for future depth, width, and length calculations. For example, measurement of the position of a navigated tool is used to determine angulation of trajectory and location on patient. This captured pose can be used as the base for all gestures in subsequent stages. If planning pre-incision, the depth of this selection is not be relevant and can be updated in the next stage.

In a depth stage, the system uses gestures to change a planned depth of the implant. In an example, the system uses the gestures to change only the depth and no other property of the implant. For example, the gestures can be used to change a position of an implant along the trajectory line defined in the trajectory stage. In an example, the system can use movements with one degree of freedom to cause a one degree of freedom change in depth. For example, a one degree of freedom measurement of positioning of the navigated tool using an augmented gesture to modify depth of captured pose.

In a width stage, the system uses detected gestures to change an implant width. In an example, the system uses the gestures to change only the width of the implant and no other property of the implant. For example, one degree of freedom measurements of the navigated tool using an augmented gesture

In a length stage, a system uses detected gestures to change an implant length. In an example, the system uses the gestures to change only the length of the implant and no other property of the implant. For example, one degree of freedom measurements of the navigated tool using an augmented gesture.

Example one degree of freedom gestures that can be used are: cranial-caudal translation of the instrument relative to patient anatomy from current position, medial-lateral (e.g., left-right) translation of the instrument relative to the patient anatomy from the current position, rotation about an instrument axis of the instrument, rotation about a cranial-caudal axis of patient anatomy from current position, or rotation about the medial-lateral axis of patient anatomy from current position.

Each of the augmented gestures can be scaled in terms of motion from current position. For example, X mm of motion results in a scaled Y mm of motion of the captured pose along one axis, X mm of motion results in a scaled Y degrees of motion of the captured pose about one axis, X degrees of motion results in a scaled Y mm of motion of the captured pose along one axis, X degrees of motion results in a scaled Y degrees of motion of the captured pose about one axis, X mm of motion results in a discrete selection of options, X degrees of motion results in a discrete selection of options, other options, or combinations thereof.

During screw placement, a working corridor is created using a cannulated instrument such as a dilator, sleeve or bushing. This instrument is typically either pre-assembled to bone prep instruments (e.g., taps, drills, burs, screwdrivers, other instruments, or combinations thereof) or used as stand-alone instruments that are manually inserted and removed during use. These solutions add additional steps during the procedure.

Example technology described herein improves efficiency in use of hollow instruments. Method of automatic retention and removal of a sleeve or dilator that creates a working corridor during implant (e.g., screw) placement in spine or other surgery.

In an example implementation, the outside diameter of an instrument (e.g., a sleeve, dilator, or bushing) is featured with an undercut and a ramped surface to facilitate insertion and automatic locking. The locking ability is provided by a spring-loaded button that slides over the ramp during insertion and engages the undercut on the mating instrument during extraction. Once locked, the two instruments become a single assembly that is removed by the surgeon with a single step.

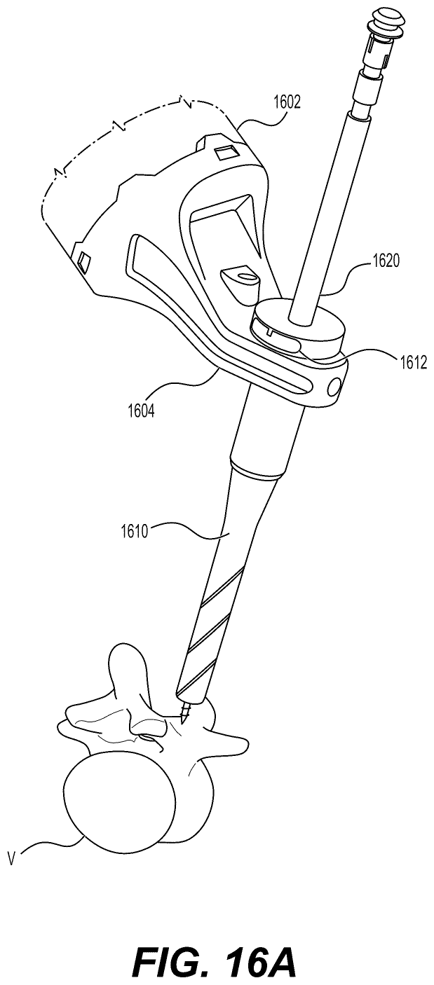

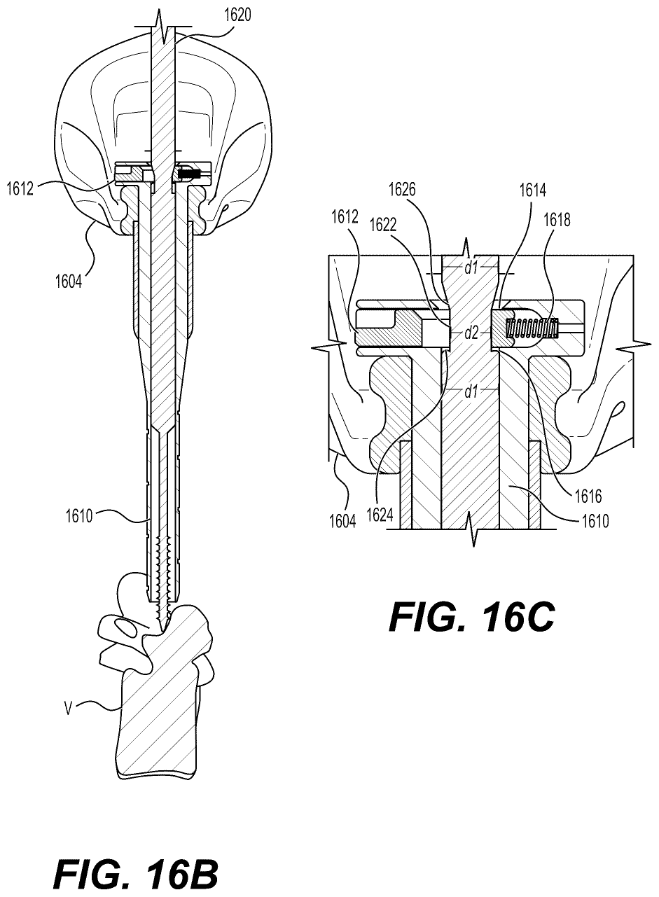

FIG. 16A illustrates a robot arm 1602 having a robot end effector 1604 coupled thereto and disposed proximate a vertebra V. Within the robot end effector 1604 is a sleeve 1610 having a button 1612. Within the sleeve 1610 is an instrument 1620. Here as specifically illustrated, the instrument 1620 is a tap.

In another example, the instrument 1620 is a dilator. This arrangement can be achieved before or after an incision is made. In some examples, the sleeve 1610 is inserted into the end effector 1604 first and then the instrument 1620 is inserted into the sleeve 1610. In another example, the instrument 1620 is inserted into the sleeve 1610 and then inserted into the end effector 1604. The combination can be used to create a working lumen through soft tissue of the recipient to the vertebra V.

In other examples, the instrument 1620 can be a burr. The sleeve 1610 can be or act as a dilator or soft shield to protect tissue or to provide insulation for the use of neuromonitoring electrodes.

The sleeve 1610 and the instrument 1620 can be so configured that the instrument has a region that the sleeve 1610 catches on. The instrument 1620 can be inserted into the sleeve 1610 up to the catch (and in some instances beyond), but the catching is such that withdrawal of the sleeve 1610 causes withdrawal of the instrument 1620 and vice versa. This catching can be achieved in any of a variety of ways. One example is shown in FIG. 16B.

FIG. 16B illustrates a cross section view of FIG. 16A robot end effector 1604, sleeve 1610, button 1612, and instrument 1620. FIG. 16C illustrates an enlarged view of the button 1612 region of FIG. 16B.

The button 1612 can include a ramp 1614 and abutment 1616. The sleeve 1610 can include a biaser 1618 (e.g., a spring) that biases the button 1612 outward.

As illustrated, the instrument 1620 has a retention groove 1622 that is a region of decreased diameter d2 compared to the diameter d1 of adjacent portions of the instrument 1620. The distal end of the groove 1622 can be bounded by a catch 1624. The catch 1624 can be a region of sharp change in diameter that forms a region that the abutment 1616 of the button 1612 interferes with such that retraction of the instrument 1620 causes the catch 1624 to press against the abutment 1616. This pressing can causes the instrument 1620 and sleeve to be removed simultaneously.

In some examples, the proximal end of the groove 1622 can be bounded by a catch as well. In the illustrated example, however, the proximal end of the groove 1622 is bound by a ramp 1626 such that the instrument 1620 can be inserted into the sleeve 1610 past the groove 1622 and the sleeve 1610 only substantially interferes with the movement of the instrument 1620 when a user attempts to withdraw the instrument 1620 past the button 1612.

When there is no object in the lumen of the sleeve 1610, the biaser 1618 urges the button 1612 outward such that the ramp 1614 and abutment 1616 of the button 1612 at least partially obstruct the lumen of the sleeve 1610. The instrument 1620 can be inserted into the sleeve 1610. As the distal end of the instrument 1620 presses the ramp 1626, the force causes the button 1612 and its components to move out of the way to permit the instrument 1620 to pass. The instrument 1620 continues to pass through or within the button 1612 and the lumen. While the instrument 1620 passes therethrough, the biaser 1618 pushes at least a portion of the button 1612 against the diameter of the instrument 1620. When the groove 1622 reaches the button 1612, the biaser 1618 pushes at least a portion of the button 1612 into the groove. At this point, the instrument 1620 is locked with the sleeve 1610.

A user can then withdraw the sleeve 1610 and the instrument 1620 simultaneously by withdrawing the instrument 1620 alone. A user can also selectively remove just the instrument 1620 by pressing the button 1612 while removing the instrument 1620. By sufficiently pressing the button 1612, the abutment 1616 is moved out of the way so it does not contact the catch 1624 in a way that would restrict withdrawal of the instrument 1620.

In other implementations, the instrument 1620 lacks a groove 1622. Instead, the sleeve 1610 catches the instrument 1620 by the button 1612 sufficient force (e.g., from the biaser 1618) and friction to catch on the instrument 1620.

Example techniques for implementing such computer functions include frameworks and technologies offering a full stack of plug-and-play capabilities for implementing desktop and browser-based applications (e.g., the applications implementing aspects described herein). The frameworks can provide a desktop web application featuring or using an HTTP server such as NODEJS or KATANA and an embeddable web browser control such as the CHROMIUM EMBEDDED FRAMEWORK or the JAVA/.NET CORE web view. The client-side frameworks can extend that concept by adding plug-and-play capabilities to desktop and the web shells for providing apps capable of running both on the desktop and as a web application. One or more components can be implemented using a set of OWIN (Open Web Interface for .NET) components built by MICROSOFT targeting the traditional .NET runtime.

KATANA, and by definition OWIN, allow for chaining together middleware (OWIN-compliant modules) into a pipeline thus offering a modular approach to building web server middleware. For instance, the client-side frameworks can use a Katana pipeline featuring modules such as SIGNALR, security, an HTTP server itself. The plug-and-play capabilities can provide a framework allowing runtime assembly of apps from available plugins. An app built atop of a plug-and-play framework can have dozens of plugins, with some offering infrastructure-level functionality and other offering domain-specific functionality. The CHROMIUM EMBEDDED FRAMEWORK is an open source framework for embedding the CHROMIUM browser engine with bindings for different languages, such as C# or JAVA. OWIN is a standard for an interface between .NET web applications and web servers aiming at decoupling the relationship between ASP.NET applications and IIS by defining a standard interface.

A person of skill in the art, with the benefit of the disclosure herein, can use any available technologies to implement aspects described herein. These technologies include any of a variety of programming integrated development environments (e.g., MICROSOFT VISUAL STUDIO, MICROSOFT VISUAL STUDIO CODE, ECLIPSE IDE by the ECLIPSE FOUNDATION, JUPYTER notebooks, other IDEs, or combinations thereof). Further, the person of skill in the art may use artificial intelligence tools to facilitate the implementation of techniques described herein, such as GITHUB COPILOT by GUITHUB, CHATGPT by OPENAI, other tools, or combinations thereof.

Further example techniques for implementing such computer functions or algorithms include frameworks and technologies provided by or in conjunction with programming languages and associated libraries. For example, languages such as C, C++, C#, PYTHON, JAVA, JAVASCRIPT, RUST, assembly, HASKELL, other languages, or combinations thereof can be used. Such languages can include or be associated with one or more standard libraries or community provided libraries. Such libraries in the hands of someone skilled in the art can facilitate the creation of software based on descriptions herein, including the receiving, processing, providing, and presenting of data. Example libraries for PYTHON and C++ include OPENCV (e.g., which can be used to implement computer vision and image processing techniques), TENSORFLOW (e.g., which can be used to implement machine learning and artificial intelligence techniques), and GTK (e.g., which can be used to implement user interface elements). Further examples include NUMPY for PYTHON (e.g., which can be used to implement data processing techniques). In addition, other software can provide application programming interfaces that can be interacted with to implement one or more aspects described herein. For example, an operating system for the computing environment (e.g., WINDOWS by MICROSOFT CORP., MACOS by APPLE INC., or a LINUX-based operating system such as UBUNTU by CANONICAL LTD.) or another component herein (e.g., an operating system of a robot, such as IIQKA.OS or SUNRISE.OS by KUKA ROBOTICS CORPORATION where the robot is a model of KUKA ROBOTICS CORPORATION) can provide application programming interfaces or libraries to usable to implement aspects described herein. As a further example, a provider of a navigation system, laser console, wireless card, display, motor, sensors, or another component may not only provide hardware components (e.g., sensor, a camera, wireless card, motor, or laser generator), but also software components (e.g., libraries, drivers, or applications) usable to implement features with respect to the components.

1. A system comprising:
a robot arm having a tracking section;
a first fiducial array at the tracking section having first fiducials; and
a second fiducial array at the tracking section having second fiducials.

2. The system of claim 1, wherein the tracking section has a frustoconical shape with a smaller diameter end of the frustconical shape being distal to a larger diameter end of the frustconical shape.

3. The system of claim 1, wherein the first and second fiducials are infrared emitters.

4. The system of claim 1, wherein bounding boxes of the first and second fiducial arrays overlap.

5. The system of claim 1,
wherein the tracking section includes a first receptacle; and
a first pod having a first housing sized to fit within the first receptacle,
wherein the first pod has the first fiducial array.

6. The system of claim 5,
wherein the first housing contains a circuit board,
wherein the first fiducials are electrically coupled to the circuit board.

7. The system of claim 6, wherein the first fiducials are mounted to the circuit board.

8. The system of claim 6, wherein the circuit board is electrically coupled to a connector configured to electrically connect the circuit board with an electronic component of the robot arm.

9. The system of claim 5, wherein the first pod includes at least one guide pin configured to facilitate placement of the first pod within the first receptacle.

10. The system of claim 1, wherein at least one of the first fiducials is disposed within a hollow.

11. The system of claim 10, wherein the hollow has a shape of a truncated cone.

12. The system of claim 10, wherein the hollow defines an upper boundary and a lower boundary between which one or more walls extend, wherein at least one of the first fiducials is disposed at the lower boundary.

13. The system of claim 1,
wherein the first fiducials are aimed at a first focal point; and
wherein the second fiducials are aimed at a second focal point different from the first focal point.

14. The system of claim 1,
wherein each of the first and second fiducials is associated with a respective fiducial normal extending therefrom; and
wherein the fiducial normals of the first fiducials converge toward first focal point; and
wherein the fiducial normals of the second fiducials converge toward a second focal point.

15. The system of claim 14,
wherein at least one of the first fiducials is disposed within a respective hollow configured such that the respective fiducial normal of the at least one first fiducial is aimed at the first focal point.

16. The system of claim 1, wherein the robot arm further has at least one capture area configured to resist distal movement of an anchor of a drape covering the robot arm.

17. The system of claim 16, wherein the capture area defines a lead-in groove and a pocket.

18. The system of claim 16, further comprising a sensor configured to detect the presence or absence of an anchor within the capture area.

19. The system of claim 18, wherein the sensor is an optical sensor.

20. The system of claim 1, further comprising:
an activation sensor; and
a controller configured to selectively activate the first fiducial array and the second fiducial array based on output of the activation sensor and a schedule.
